SEQUENCE LISTING (6 sequences)

SEQ ID NO: 1 - protein, 451 aa, synthetic construct
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 2 - protein, 217 aa, synthetic construct
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 123 aa, synthetic construct [sequence omitted]

SEQ ID NO: 4 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 5 - protein, 110 aa, synthetic construct [sequence omitted]

SEQ ID NO: 6 - protein, 107 aa, synthetic construct [sequence omitted]

(19) United States
(12) Patent Application Publication
(10) Pub. No.: US 2025/0236662 A1
(43) Pub. Date: Jul. 24, 2025

(54) DOSAGE AND ADMINISTRATION REGIMEN FOR THE TREATMENT OR PREVENTION OF GUILLAN-BARRÉ SYNDROME BY THE USE OF THE ANTI-C5 ANTIBODY CROVALIMAB

(71) Applicants: Hoffmann-La Roche Inc., Little Falls, NJ (US); Chugai Seiyaku Kabushiki Kaisha, Tokyo (JP)

(72) Inventors: Simon Bertrand Marie BUATOIS, Basel (CH); Keisuke GOTANDA, Tokyo (JP); Kenji SHINOMIYA, Tokyo (JP); Alexandre Antoine Bernard SOSTELLY, Saint Louis (FR); Antoine Paul Maxence SOUBRET, Huningue (FR)

(73) Assignees: Hoffmann-La Roche Inc., Little Falls, NJ (US); Chugai Seiyaku Kabushiki Kaisha, Tokyo (JP)

(21) Appl. No.: 18/905,654

(22) Filed: Oct. 3, 2024

Related U.S. Application Data
(63) Continuation of application No. PCT/EP2023/058608, filed on Apr. 3, 2023.

(30) Foreign Application Priority Data
Apr. 4, 2022 (EP) 22166585.4

(51) Int. Cl. C07K 16/18; A61K 39/00; A61P 37/06

(52) U.S. Cl. CPC C07K 16/18; A61P 37/06; A61K 2039/507; A61K 2039/545; C07K 2317/92

(57) ABSTRACT

The present invention relates to a dosage and administration regimen of anti-C5 antibodies, particularly of the anti-C5 antibody Crovalimab, for use in a method of treating or preventing GBS in a subject. The dosage and treatment regimen of the present invention include the administration of an anti-C5 antibody, preferably of the anti-C5 antibody Crovalimab, with loading dose followed by the administration of (a) maintenance dose(s) of the anti-C5 antibody to the subject, wherein the initial administered loading dose is intravenously given to the subject and the maintenance doses are subcutaneously administered in a lower dosage as the intravenously administered loading dose.

### CROSS-REFERENCE TO RELATED APPLICATIONS

This application is a continuation of Patent Cooperation Treaty Application No. PCT/EP2023/058608, filed Apr. 3, 2023, which claims priority to European Application No. 22166585.4, filed Apr. 4, 2022, both of which are incorporated herein by reference in their entireties.

### SEQUENCE LISTING

The instant application contains a Sequence Listing which has been submitted electronically in XML format and is hereby incorporated by reference in its entirety. Said XML copy, created on Sep. 30, 2024, is named “P37481-US_Sequence Listing.xml” and is 7,629 bytes in size.

### FIELD OF THE INVENTION

The present invention relates to a dosage and administration regimen of anti-C5 antibodies, particularly of the anti-C5 antibody Crovalimab, for use in a method of treating or preventing Guillain-Barré Syndrome (GBS). The dosage and treatment regimen of the present invention include the administration of an anti-C5 antibody, preferably of the anti-C5 antibody Crovalimab, with loading doses followed by the administration of (a) maintenance dose(s) of the anti-C5 antibody to the subject, wherein the initial administered loading dose is intravenously given to the subject and the doses are subcutaneously administered in a lower dosage as the intravenously administered loading dose.

### BACKGROUND OF THE INVENTION

The classical pathway is normally activated by the formation of antigen-antibody complexes. Independently, the first step in activation of the lectin pathway is the binding of specific lectins such as mannan-binding lectin (MBL), H-ficolin, M-ficolin, L-ficolin and C-type lectin CL-11. In contrast, the alternative pathway spontaneously undergoes a low level of turnover activation, which can be readily amplified on foreign or other abnormal surfaces (bacteria, yeast, virally infected cells, or damaged tissue). These pathways converge at a point where complement component C3 is cleaved by an active protease to yield C3a and C3b.

Guillain-Barré syndrome (GBS) is a rare, but potentially fatal disease of the peripheral nerves and nerve roots that is usually triggered by infections. GBS is an acute, heterogeneous, paralysing, inflammatory peripheral nerve disease characterized by rapidly progressive, symmetrical limb weakness which results in muscles having reduced or even absence of response to stimuli [1].

The prognosis of GBS is determined by the extent of axonal loss in the acute phase and if axonal damage is minimized by effective early treatment in the acute phase, sufficient nerve regeneration and collateral sprouting from surviving motor axons leading to long-term recovery could be expected several months after the disease peak. Intravenous immunoglobulin (IVIg) (400 mg/kg body weight daily for 5 days) and plasma exchange (PE) (50 mL plasma/kg body weight in five sessions over 1-2 weeks), both introduced in the 1980s are established treatments for GBS, and are considered equally effective as first-line treatments. PE and IVIg were shown [2,3,4] to speed up recovery in the acute and subacute phases of the disease started within 2 weeks of the onset of weakness. Beyond these time periods, evidence on efficacy is lacking and it is still unclear whether these treatments sufficiently improve long-term outcomes in patients with Guillain-Barré syndrome [3,4]. As IVIg is easier to administer and generally more widely available than plasma exchange, it is usually the treatment of choice. Inhibition of complement is a novel approach for treating GBS; complement activation appear to contribute to nerve degeneration in GBS. Early acute treatment may prevent complement mediated long-term nerve damage. Complement activation is thought to play an important role in the pathogenesis of all GBS variants [5]. Human proof of concept data supporting the use of C5 complement inhibitor (eculizumab) are available from phase 2 JET study in patients with severe GBS [6]. This study was a 24 week, multi-center, double-blind, placebo-controlled, randomized phase 2 trials conducted in Japan. In this study, the primary outcome, the proportion of patients regaining the ability to walk by week 4, did not exceed the predefined response threshold (50%) in the eculizumab group. However, eculizumab showed potential evidence of improving motor function in the secondary endpoints [6]. Crovalimab is a novel humanized anti-C5 monoclonal antibody [7] which binds to complement protein C5 with high affinity, thereby inhibiting its cleavage to C5a and C5b and preventing the generation of the terminal complement complex C5b-9 (MAC). Crovalimab has been demonstrated [8] to inhibits terminal complement-mediated intravascular hemolysis in patients with Paroxysmal Nocturnal Hemoglobinuria (PNH).

Crovalimab is based on SMART-Ig (Recycling Antibody™) technology [7] with pH-dependent antigen binding. It provides efficient target disposal and enhanced neonatal fragment crystallizable receptor (FcRn) binding, improved antibody recycling efficiency resulting in prolonged half-life and complement inhibition. In addition, the physicochemical properties of crovalimab support the development of high concentration formulation. The combination of the SMART-Ig and the high concentrated formulation enables every 4 weeks (Q4W) SC dosing.

The half-life of IVIg and crovalimab are dependent on the recycling by FcRn receptors in the endosome, the impact of the co-administration of IVIg on crovalimab PK taking into account the binding competition of both molecules to the FcRn receptors for maintaining the C5 inhibition. over a 28 day period.

### SUMMARY OF THE INVENTION

This need is addressed by the present invention by providing the embodiments as defined in the claims.

The present invention relates to an anti-C5 antibody for use in a method of treating or preventing a GBS in a subject, wherein the method comprises the consecutive steps of:
(a) intravenously administering a loading dose of 1500 mg of the anti-C5 antibody to the subject once,
(b) followed by subcutaneously administering at least one maintenance dose of 340 mg of the anti-C5 antibody to the subject.

In the context of the present invention, the subject to be treated is preferably a patient with a body weight above 100 kg. In the context of the present invention the subject to be treated is/are subject/s which suffer from GBS.

Moreover, the invention is directed to the use of the anti-C5 antibody for the treatment or prevention of GBS. In the context of the present invention, the present invention is directed to the treatment or prevention of GBS, in patients that are treated with a combination of an anti-C5 antibody, preferably Crovalimab, and the Standard of Care (SOC) Intravenous Immunoglobulin (IVIg). IVIg is a pool of immunoglobulins from the plasma of healthy donors prepared by separating the immunoglobulins from the other components of the plasma. Examples of IVIg are Asceniv, Bivigam, Carimune, Cutaquig, Cuvitru, Flebogamma, Gammagard, GamaSTAN, Gammaked, Gammaplex, Gamunex-C, Hizentra, Hyqvia, Octagam, Panzyga, Privigen, Xembify. Accordingly, the herein described dosage and administration regimen of the anti-C5 antibody, particularly of the anti-C5 antibody Crovalimab, is given to patients who is treated with a combination of anti-C5 antibody, preferably Crovalimab, and IVIg.

Accordingly, the present invention relates to an anti-C5 antibody, preferably the anti-C5 antibody Crovalimab, for use in a method of treating or preventing a GBS in a subject, preferably a subject with a body weight above 100 kg, wherein the method comprises the consecutive steps of:
(a) intravenously administering a loading dose of 1500 mg of the anti-C5 antibody to the subject once,
(b) followed by subcutaneously administering at least one loading dose of 340 mg of the anti-C5 antibody to the subject.

The “loading dose” refers to the dose of the anti-C5 antibody administered to the subject suffering from GBS, at the beginning of the treatment, i.e. at the start of the treatment regimen. In pharmacokinetics (PK), a “loading dose” is an initial higher dose of a drug that may be given to a patient at the beginning of a course of treatment before dropping down to a lower dose. In the context of the present invention, the loading dose is firstly given to subjects to be treated by intravenous administration. In the context of the present invention, the loading dose is given once at a dose of 1500 mg. Accordingly, in the context of the present invention, a loading dose of a composition formulated for intravenous administration is given intravenously once to the subject before one dose or more doses of a pharmaceutical composition formulated for subcutaneous administration is/are given subcutaneously.

According to the present invention, the initial dose is followed by subsequent doses of equal or smaller amounts of anti-C5 antibody at intervals sufficiently close to maintain the concentration of the anti-C5 antibody at or above an efficacious target level. Accordingly, in the context of the present invention, (a) maintenance dose(s) is (are) administered to the patients after the loading dose. The “maintenance dose” refers to the dose of the anti-C5 antibody that is given to a subject suffering from a C5-related disease to maintain the concentration of the anti-C5 antibody above a certain efficacious threshold of the anti-C5 antibody concentration during the treatment period. In the context of the present invention the target level of the anti-C5 antibody is a median of approximately 100 μg/ml or more over the treatment period. The target level of the anti-C5 concentration within the present invention may be determined in a biological sample of the subject to be treated. Means and methods for the determination of the anti-C5 concentration in a biological sample are within the common knowledge of the skilled person and can for example be determined by an immunoassay. Preferably in the context of the present invention, the immunoassay is an ELISA. Preferably, the maintenance dose(s) is (are) subcutaneously administered to the patients, at a dose or doses of 340 mg of the anti-C5 antibody. Accordingly, within the context of the present invention at least one maintenance, or more maintenance doses is/are given to the subject, wherein the maintenance dose(s) is (are) subcutaneously administered at a dose of 340 mg. In the context of the present invention, at least one maintenance dose of 340 mg of the anti-C5 antibody is administered to the patients after the intravenous administration of a loading dose of 1500 mg of the anti-C5 antibody. The subcutaneously administered dose(s) is (are) subcutaneously administered at a dose of 340 mg of the anti-C5 antibody at least once to the subject 1 day to 3 weeks (21 days) after the start of the intravenous administration of the anti-C5 antibody. Accordingly, in the context of the present invention, a dose of 340 mg of the anti-C5 antibody is subcutaneously administered at least once to the subject 1, 2, 3, 4, 5, 6, 7, 8, 9, 10, 11, 12, 13, 14, 15, 16, 17, 18, 19, 20, or 21 days after the start of the intravenous administration of the anti-C5 antibody. Preferably, a dose of 340 mg of the anti-C5 antibody is administered to the subject 1 day after the start of the intravenous administration of the anti-C5 antibody. More preferably, one dose of 340 mg of the anti-C5 antibody is subcutaneously administered 1 day after the start of the intravenous administration. In the context of the present invention, at least one additional dose of 340 mg of the anti-C5 antibody is subcutaneously administered to the subject 1 week (7 days), 2 weeks (14 days), or 3 weeks (21 days) after the start of the intravenous administration of the anti-C5 antibody. Most preferably, additional doses of 340 mg of the anti-C5 antibody are subcutaneously administered 1 week (7 days), 2 weeks (14 days) and 3 weeks (21 days) after the start of the intravenous administration of the anti-C5 antibody. Accordingly, within the context of the present invention 1, 2, 3, 4 and/or 5 doses is/are given to the subject, wherein the loading dose, is intravenously administered at a dose of 1500 mg to the subject, and wherein 1, 2, 3 or 4 doses is/are given subcutaneously at a dose of 340 mg to the patient. In the context of the present invention, the subcutaneous administration of 4 loading doses each having a dosage of 340 mg of the anti-C5 antibody is preferred, wherein the additional doses are subcutaneously administered once 1 day after the start of the intravenous administration of the anti-C5 antibody, followed by subcutaneous administration of maintenance doses 1 week, 2 weeks and 3 weeks once weekly after the start of the intravenous administration of the anti-C5 antibody. For example, the total amount of the anti-C5 antibody given via (a) maintenance dose(s) corresponding to an intravenous administration of 1500 mg (day 1), followed by subcutaneous administration of 340 mg (day 2), 340 mg (day 8), 340 mg (day 15) and 340 mg (day 22) is 2860 mg.

In particular, the present invention relates to an anti-C5 antibody for use in a method of treating or preventing a C5-related disease in a subject, preferably in a subject with a body weight above 100 kg, wherein the method comprises the consecutive steps of:
(i) intravenously administering a loading dose of 1500 mg of the anti-C5 antibody to the subject once;
(ii) subcutaneously administering a maintenance dose of 340 mg of the anti-C5 antibody to the subject 1 day after the start of the intravenous administration of the anti-C5 antibody;
(iii) subcutaneously administering a maintenance dose of 340 mg of the anti-C5 antibody to the subject 1 week (7 days), 2 weeks (14 days) and 3 weeks (21 days) after the start of the intravenous administration of the anti-C5 antibody once weekly.

The terms “intravenous administration”/“intravenously administering” refer in the context of the present invention to the administration of the anti-C5 antibody into a vein of the subject such that the body of the patient to be treated receives the anti-C5 antibody in approximately 15 minutes or less, preferably 5 minutes or less. For intravenous administration, the anti-C5 antibody has to be formulated that it be administered via a suitable device such as (but not limited to) a syringe. In the context of the present invention, the formulation for intravenous administration comprises 50 to 350 mg of the anti-C5 antibody, 1 to 100 mM of a buffering agent, such as histidine/aspartic acid comprising a pH of 5.5±1.0, 1 to 100 mM of an amino acid such as arginine, and 0.01 to 0.1% of a non-ionic surfactant, such as a poloxamer. Preferred in the context of the present invention, the formulation for intravenous administration is provided in a 2 mL glass vial containing the following components: 170 mg/ml Crovalimab, 30 mM histidine/aspartic acid (pH 5.8), 100 mM arginine hydrochloride and 0.05% Poloxamer 188™. The formulation is then administered to the patient within a tolerated time period, such as 5 minutes, 15 minutes, 30 minutes, 90 minutes, or less. Moreover, the formulation for intravenous administration is given to the patients to be treated with an injection volume of between 1 ml to 15 ml, preferably about 6 ml.

The terms “subcutaneous administration”/“subcutaneously administering” refer in the context of the present invention to the introduction of the anti-C5 antibody under the skin of an animal or human patient, preferable within a pocket between the skin and underlying tissue, by relatively slow, sustained delivery from a drug receptacle. The pocket may be created by pinching or drawing the skin up and away from underlying tissue. For subcutaneous administration, the anti-C5 antibody has to be formulated that it may be administered via a suitable device such as (but not limited to) a syringe, a prefilled syringe, an injection device, an infusion pump, an injector pen, a needless device, or via a subcutaneous patch delivery system. In the context of the present invention, the formulation for subcutaneous administration comprises 50 to 350 mg of the anti-C5 antibody, 1 to 100 mM of a buffering agent, such as histidine/aspartic acid comprising a pH of 5.5±1.0, 1 to 100 mM of an amino acid such as arginine, and 0.01 to 0.1% of a non-ionic surfactant, such as a poloxamer. Preferred in the context of the present invention, the formulation for intravenous administration is provided in a 2.25 prefilled syringe containing the following components: 170 mg/ml Crovalimab, 30 mM histidine/aspartic acid (pH 5.8), 100 mM arginine hydrochloride and 0.05% Poloxamer 188™. In the context of the present invention a formulation for the subcutaneous administration is provided in a prefilled syringe with a needle safety device. The injection devices for subcutaneous administration comprises about 1 to 15 ml or more, preferably 2.25 ml of a formulation for subcutaneous administration comprising the anti-C5 antibody. Under normal circumstances, the injection volume to be subcutaneously administered is 1 to 15 ml, preferably either 2 ml (340 mg Crovalimab), or 4 ml (680 mg Crovalimab). In the context of the present invention, the subcutaneous administration refers to introduction of the anti-C5 antibody under the skin of the patient to be treated by relatively slow, sustained delivery from a drug receptacle for a period of time including, but not limited to, 30 minutes or less, 90 minutes or less. Optionally, the administration may be made by subcutaneous implantation of a drug delivery pump implanted under the skin of the patient to be treated, wherein the pump delivers a predetermined amount of the anti-C5 antibody for a predetermined period of time, such as 30 minutes, 90 minutes, or a time period spanning the length of the treatment regimen.

In the context of the present invention the above dosages and treatment regimens can be useful for the treatment or prevention of GBS in a subject to whom is IVIg is co-administrated. For example, the treatment regimen of the present invention can be useful for treating a patient having GBS, wherein the patient also receives the Standard of Care. Preferably the SOC is intravenous administration of IVIg.

The present invention also relates to a pharmaceutical composition for use in combination with IVIg for treating or preventing GBS, the composition comprises an anti-C5 antibody, preferably Crovalimab, and is administered by the following administration steps:
(a) intravenously administering a loading dose of 1500 mg of the anti-C5 antibody to the subject once; and
(b) subcutaneously administering at least one maintenance dose of 340 mg of the anti-C5 antibody to the subject.

In one embodiment, wherein
(i) 1500 mg of the anti-C5 antibody is intravenously administered to the subject (loading dose) once;
(ii) 340 mg of the anti-C5 antibody is subcutaneously administered of to the subject 1 day after the start of the intravenous administration of the anti-C5 antibody (maintenance dose)

In a further embodiment, the pharmaceutical composition comprising IVIg is administered by the following steps:
(a) 400 mg/kg of IVIg is intravenously administered once to the subject on the same day as the loading dose of the C5 antibody;
(b) 400 mg/kg of IVIg is intravenously administered to the subject daily 1 day, 2 days, 3 days and 4 days after the start of the intravenous administration of the anti-C5 antibody.

The present invention also relates to a pharmaceutical composition for use in combination with an anti-C5 antibody, preferably Crovalimab, for treating or preventing GBS, the composition comprises IVIg and is administered by the following administration steps:
(a) intravenously administered to the subject once on the same day as the loading dose of the C5 antibody (1500 mg)
(b) intravenously administered to the subject daily 1 day, 2 days, 3 days and 4 days after the start of the intravenous administration of the anti-C5 antibody.

In one embodiment the intravenously administered dose of IVIg of a) is 400 mg/kg. In a further embodiment, intravenously administered dose of IVIg 2 days, 3 days and 4 days after the start of the intravenous administration of the anti-C5 antibody (step b) is 400 mg/kg daily.

In a further embodiment, the pharmaceutical composition comprising the anti-C5 antibody is administered by the following steps:
(i) 1500 mg of the anti-C5 antibody is intravenously administered to the subject (loading dose) once;
(ii) 340 mg of the anti-C5 antibody is subcutaneously administered of to the subject 1 day after the start of the intravenous administration of the anti-C5 antibody (maintenance dose).

In particular, the present invention relates to a pharmaceutical composition comprising an anti-C5 antibody for use in a method of treating or preventing GBS in a subject, preferably in a subject with a body weight above 100 kg, wherein the method comprises the steps of:
(i) intravenously administering a loading dose of 1500 mg of the anti-C5 antibody to the subject once;
(ii) subcutaneously administering a maintenance dose of 340 mg of the anti-C5 antibody to the subject 1 day after the start of the intravenous administration of the anti-C5 antibody;
(iii) subcutaneously administering a maintenance dose of 340 mg of the anti-C5 antibody to the subject 1 week (7 days), 2 weeks (14 days) and 3 weeks (21 days) after the start of the intravenous administration of the anti-C5 antibody once weekly;
wherein the anti-C5 antibody is used in combination with IVIg, wherein
   (a) 400 mg/kg of IVIg is intravenously administered once to the subject on the same day as the loading dose of the C5 antibody of (i)
   (b) 400 mg/kg of IVIg is intravenously administered to the subject daily 1 day, 2 days, 3 days and 4 days after the start of the intravenous administration of the anti-C5 antibody.

The administration of initial doses of the anti-C5 antibody and IVIg may be administered together or separated. If anti-C5 antibody and IVIg are administered separately, they may administered directly subsequent one another or timely spaced. For example, the loading does of the anti-C5 antibody is given first, directly followed by the loading dose of the IVIg or, the IVIg loading dose may be administered first, directly followed by the loading of the anti-C5 antibody. Alternatively, two loading doses may be administered timely spaced, for example the two loading doses may be administered separated by time period from 5, 10, 15, 20, 30, 40, 50, 60 minutes; up to 2, 3, 4, 5, 6, 7, 8, 9, 10 hours, or 1 to 23 hours, 1 to 16 hours, 1 to 8 hours, 1 to 4 hours, 1 to 2 hours. For example the loading dose of the anti-C5 antibody is administered in the morning and the first dose of IVIg is administered in the evening, or first dose of IVIg is administered in the morning and the loading dose of the anti-C5 antibody is administered in the evening.

The present invention also relates to a combination of an anti-C5 antibody and IVIg for use in a method of treating or preventing a GBS in a subject, preferably in a subject with a body weight above 100 kg, wherein the method comprises the consecutive steps of:
(i) intravenously administering a loading dose of 1500 mg of the anti-C5 antibody to the subject once and intravenously administering 400 mg/kg of IVIg once on the same day;
(ii) subcutaneously administering a maintenance dose of 340 mg of the anti-C5 antibody to the subject 1 day after the start of the intravenous administration of the anti-C5 antibody and intravenously administering 400 mg/kg of IVIg on the same day;
(iii) intravenously administering 400 mg/kg of IVIg daily 2 days, 3 days and 4 days after the start of the intravenous administration of the anti-C5 antibody
(IV) subcutaneously administering a maintenance dose of 340 mg of the anti-C5 antibody to the subject 1 week (7 days), 2 weeks (14 days) and 3 weeks (21 days) after the start of the intravenous administration of the anti-C5 antibody once weekly.

The administration of initial doses of the anti-C5 antibody and IVIg may be administered together or separated. If anti-C5 antibody and IVIg are administered separately, they may administered directly subsequent one another or timely spaced. For example, the loading does of the anti-C5 antibody is given first, directly followed by the loading dose of the IVIg or, the IVIg loading dose may be administered first, directly followed by the loading of the anti-C5 antibody. Alternatively, two loading doses may be administered timely spaced, for example the two loading doses may be administered separated by time period from 5, 10, 15, 20, 30, 40, 50, 60 minutes; up to 2, 3, 4, 5, 6, 7, 8, 9, 10 hours, or 1 to 23 hours, 1 to 16 hours, 1 to 8 hours, 1 to 4 hours, 1 to 2 hours. For example the loading dose of the anti-C5 antibody is administered in the morning and the first dose of IVIg is administered in the evening, or first dose of IVIg is administered in the morning and the loading dose of the anti-C5 antibody is administered in the evening.

In the context of the present invention, a “week” refers to a period of time of 7 days.

In the context of the present invention, a “month” refers to a period of time of 4 weeks.

“Treatment” in the context of the present invention comprises the sequential succession of an “induction treatment” and at least a “maintenance treatment”. Typically, a treatment according to the invention comprises an “induction treatment” and at least one “maintenance treatment”. Typically, a treatment according to the invention may be 3 weeks to 1 month, e.g. 28 days.

An “induction treatment” consists in an intravenous administration of a loading dose, preferably a dose of 1500 mg, of the anti-C5 antibody to the subject. As explained herein above, a “maintenance treatment” consists in the sequential succession of (i) a maintenance period wherein one or more maintenance dose(s) is (are) subcutaneously given to the subjects. In the context of the present invention, it is preferred that a maintenance dose of 340 mg of the anti-C5 antibody is given to the subject is given 1 day, 1 week (7 days), 2 weeks (14 days) and 3 weeks (21 days) after the intravenously administered loading dose was given to the subject. Preferably, the loading dose to be administered intravenously has a dose of 1500 mg. The maintenance dose which is subcutaneously given to the subject to be treated has a dose of 1360 mg. Thus, in the context of the present invention a dose of 2360 mg of the anti-C5 antibody is either intravenously, or subcutaneously administered to the subject to be treated during the treatment period.

In the context of the present invention, the anti-C5 antibody is preferably Crovalimab. The sequence details of the anti-C5 antibody Crovalimab (CAS number: 1917321-26-6) are disclosed in List No. 119 of proposed International Non-proprietary Names for Pharmaceutical Substances (INN) as published at pages 302 and 303 of WHO Drug Information (2018), Vol. 32, No. 2. The sequences of the anti-C5 antibody Crovalimab is also shown in SEQ ID NO: 1 (heavy chain) and SEQ ID NO: 2 (light chain). The generation of the anti-C5 antibody Crovalimab used in the present invention is described in WO 2016/098356 (see Example 1.1 for details). Further, in the context of the present invention, the anti-C5 antibody Crovalimab is administered to the patients by a formulation either for intravenous administration, or for subcutaneous administration. Preferred in the context of the present invention is the intravenous or subcutaneous administration of the herein provided dosages as (a) fixed-dose(s).

The formulation for intravenous administration comprises 50 to 350 mg of the anti-C5 antibody Crovalimab, 1 to 100 mM of a buffering agent, such as histidine/aspartic acid comprising a pH of 5.5±1.0, 1 to 100 mM of an amino acid such as arginine, and 0.01 to 0.1% of a non-ionic surfactant, such as a poloxamer. Preferred in the context of the present invention, the formulation for intravenous administration is provided in a 2 mL glass vial containing the following components: 170 mg/ml Crovalimab, 30 mM histidine/aspartic acid (pH 5.8), 100 mM arginine hydrochloride and 0.05% Poloxamer 188™.

The formulation for subcutaneous administration comprises 50 to 350 mg of the anti-C5 antibody Crovalimab, 1 to 100 mM of a buffering agent, such as histidine/aspartic acid comprising a pH of 5.5±1.0, 1 to 100 mM of an amino acid such as arginine, and 0.01 to 0.1% of a non-ionic surfactant, such as a poloxamer. Preferred in the context of the present invention, the formulation for intravenous administration is provided in a 2.25 prefilled syringe containing the following components: 170 mg/ml Crovalimab, 30 mM histidine/aspartic acid (pH 5.8), 100 mM arginine hydrochloride and 0.05% Poloxamer 188™.

Patients described in the context of the present invention are patients suffering from GBS. Preferred patients in the context of the present invention are patients with a body weight above 100 kg. In the context of the present invention, patients preferably are co-administered IVIg.

Preferably, IVIg is administered in combination with an anti-C5 antibody to the subject suffering from GBS, wherein 400 mg/kg of IVIg is
(a) intravenously administered to the subject once on the same day as the loading dose of the C5 antibody (1500 mg)
(b) intravenously administered to the subject daily 1 day, 2 days, 3 days and 4 days after the start of the intravenous administration of the anti-C5 antibody

Moreover, the present invention relates to a method of treating or preventing a C5-related disease in a subject, wherein the method comprises the consecutive steps of:
(a) intravenously administering a loading dose of 1500 mg of the anti-C5 antibody to the subject once; and
(b) subcutaneously administering at least one maintenance dose of 340 mg of the anti-C5 antibody to the subject.

It is preferred in the context of the present invention that the method of treating or preventing a C5-related disease in a subject is carried out by the following administration steps:
(i) intravenously administering a loading dose of 1500 mg of the anti-C5 antibody to the subject once;
(ii) subcutaneously administering a maintenance dose of 340 mg of the anti-C5 antibody to the subject 1 day after the start of the intravenous administration of the anti-C5 antibody;
(iii) subcutaneously administering a loading dose of 340 mg of the anti-C5 antibody to the subject 1 week, 2 weeks and 3 weeks after the start of the intravenous administration of the anti-C5 antibody once weekly.

It is even more preferred in the context of the present invention that the method of treating or preventing a C5-related disease in a subject is carried out by the following administration steps:
(i) intravenously administering a loading dose of 1500 mg of the anti-C5 antibody to the subject once and intravenously administering 400 mg/kg of IVIg once on the same day;
(ii) subcutaneously administering a maintenance dose of 340 mg of the anti-C5 antibody to the subject 1 day after the start of the intravenous administration of the anti-C5 antibody and intravenously administering 400 mg/kg of IVIg on the same day;
(iii) intravenously administering 400 mg/kg of IVIg daily 2 days, 3 days and 4 days after the start of the intravenous administration of the anti-C5 antibody
(IV) subcutaneously administering a maintenance dose of 340 mg of the anti-C5 antibody to the subject 1 week (7 days), 2 weeks (14 days) and 3 weeks (21 days) after the start of the intravenous administration of the anti-C5 antibody once weekly.

As explained above, it is preferred in the context of the present invention that the anti-C5 antibody used in the context of the dosage and administration regiment is Crovalimab. Further, the definition given above apply likewise to the above methods of treating or preventing a C5-related disease. It is also preferred in the context of the present invention that the subject to be treated has a body weight above 100 kg.

### FIGURES

The Figures show:

FIG. 1 Radiolabeled IVIg pharmacokinetic in serum and total radioactivity as proportion of injected dose

FIG. 2A from Kendrick F, Evans N D, Berlanga O, Harding S J, Chappell M J. Parameter identification for a model of neonatal Fc receptor-mediated recycling of endogenous immunoglobulin G in humans. Front Immunol. 10. (2019)

FIG. 2 Mean (SEM) M281 PK profiles from single ascending dose in First-in-Human study in healthy volunteers for five different doses Figure extracted from Momenta R&D Day 2018 Presentation

FIG. 3 Average Serum IgG concentration as percentage relative to baseline (%) as function of M281 dose in Single Ascending Dose (SAD) and Multiple Ascending Dose (MAD) studies

Figure extracted from Ling L E, Hillson J L, Tiessen R G, et al. M281, an Anti-FcRn Antibody: Pharmacodynamics, Pharmacokinetics, and Safety Across the Full Range of IgG Reduction in a First-in-Human Study. Clin Pharmacol Ther. (2018)

FIG. 4 PK/PD Model in Serum for Crovalimab, C5, IgG, M281 and their Recycling in the Endosome by FcRn

Each box corresponds the concentration (nM) of the entities defined in Table 4. [Ab1], [Ab1Ag], [AgAb1Ag], [Ag], [IgG], [IgG*], [M281] are the concentrations in the central compartment. [Ab1]p, [Ab1Ag]p, [AgAb1Ag]p are the concentrations in the peripheral compartment. CLAb1 and CLAg are the clearance (L/day/kg) of the free Ab1 and free Ag. CLe, Ab1, CLe, Ab1Ag, CLe, AgAb1Ag, CLe, IgG, CLe, M281 are the clearance (L/day/kg) of Ab1, Ab1Ag, AgAb1Ag, IgG and M281 into the endosome. CLe, Ab1 recy, CLe, IgG recy, CLe, M281 recy are the clearance (L/day/kg) of Ab1, IgG and M281 from the endosome back to plasma. Ab1, IgG and M281 not bound to FcRn are eliminated with a rate defined by the elimination constant ke, Ab1, ke, IgG and ke, M281 (1/day). Vc, Vc IgG and Vc M281 are the are the volume (L/kg) of the central compartment for Ab1/Ag, IgG and M281. VpAb1, Vp Ab1Ag, Vp AgAb1Ag and Vp IgG are the volume (L/kg) of the peripheral compartments. Q Ab1 and Q IgG are the inter-compartmental clearance (L/day/kg). kinAg is the production rate (nmol/day) of Ag. konAb1 (nM/day) and koffAb1 (1/day) are the association and dissociation rate of Ab1 with Ag.

FIG. 5 ODE Equations for the Binding Model of Ab1 with Ag in Serum

FIG. 6 ODE Equations for IgG, IgG* and M281 in Serum

FIG. 7 ODE Equations for Ab1, IgG, IgG* and M281 in the Endosome

FIG. 8 Ab1 Disposition term from serum to the endosome and Ab1 recycling term from the endosome to the serum

FIG. 9 Equations for Ab1 in the Endosome

FIG. 10 Model Individual Goodness Fit for Total Ab1, Total Ag and Free Ag for COMPOSER Part 1 (Healthy Volunteer Subjects 11001-11007)

Each column corresponds to a subject and the rows to total Ab1 (aka [Ab1]total=[Ab1]+[Ab1Ag]+[AgAb1Ag]), total Ag (aka [Ag]total=[Ag]+[Ab1Ag]+2[AgAb1Ag]) and free Ag (aka [Ag]) respectively. The concentrations observed in the COMPOSER study (Example 2.1) are displayed as black dots and the continuous lines are the simulations performed using the empirical Bayes model parameter estimates for each subject.

FIG. 11 Model Individual Goodness Fit for Total Ab1, Total Ag and Free Ag for COMPOSER Part 1 (Healthy Volunteer Subjects 11008-11015)

Each column corresponds to a subject and the rows to total Ab1 (aka [Ab1]total=[Ab1]+[Ab1Ag]+[AgAb1Ag]), total Ag (aka [Ag]total=[Ag]+[Ab1Ag]+2[AgAb1Ag]) and free Ag (aka [Ag]) respectively. The concentrations observed in the COMPOSER study (Example 2.1) are displayed as black dots and the continuous lines are the simulations performed using the empirical Bayes model parameter estimates for each subject

FIG. 12 Model Individual Goodness Fit for Total Ab1, Total Ag and Free Ag for COMPOSER Part 2 (Naïve PNH Patients)

Each column corresponds to a subject and the rows to total Ab1 (aka [Ab1]total=[Ab1]+[Ab1Ag]+[AgAb1Ag]), total Ag (aka [Ag]total=[Ag]+[Ab1Ag]+2[AgAb1Ag]) and free Ag (aka [Ag]) respectively. The concentrations observed in the COMPOSER study (Example 2.1) are displayed as black dots and the continuous lines are the simulations performed using the empirical Bayes model parameter estimates for each subject.

FIG. 13 Model Individual Goodness Fit for Total Ab1, Total Ag and Free Ag for COMPOSER Part 4 (Naïve PNH Patients)

Each column corresponds to a subject and the rows to total Ab1 (aka [Ab1]total=[Ab1]+[Ab1Ag]+[AgAb1Ag]), total Ag (aka [Ag]total=[Ag]+[Ab1Ag]+2[AgAb1Ag]) and free Ag (aka [Ag]) respectively. The concentrations observed in the COMPOSER study (Example 2.1) are displayed as black dots and the continuous lines are the simulations performed using the empirical Bayes model parameter estimates for each subject.

FIG. 14 Model Individual Goodness Fit for radiolabeled IgG* in serum (upper panel), and Total Body Radioactivity normalized by the injected dose (lower panel) Each column corresponds to a subject and the rows to dose normalized IgG* in serum (i.e. [IgG*]*Vc IgG/dose), total radioactivity (i.e. [Vc IgG*[IgG*]+Vp IgG*[IgG*]p+Ve*([IgG*]e+[IgG*−FcRn]e)]/dose). The observations from the radiolabeled studies (Example 2.2) are displayed as black dots and the continuous lines are the simulations performed using the empirical Bayes model parameter estimates for each subject.

FIG. 15 Model Individual Goodness Fit for M281PK, and endogenous IgG normalized by baseline IgG level

Each column corresponds to the average data for each SAD and MAD trial arm and the rows to M281 PK (aka [M281]) and the ratio of endogenous

IgG to its baseline value (aka [IgG]/[IgG]baseline). The observations from the M281 SAD and MAD studies (Example 2.3) are displayed as black dots and the continuous lines are the simulations performed using the empirical Bayes model parameter estimates for each study arm.

FIG. 16 Simulated Median and Min/Max Time Profiles for 33 Subjects Receiving Only Crovalimab (in black) or Crovalimab with IVIg (in blue). All Concentrations are in uM

Median (continuous lines) and min/max (dotted lines) simulated time profiles for 33 subjects receiving either crovalimab alone (black lines) or crovalimab with IVIg (blue lines);

Crovalimab regimen: 1000 mg IV on Day 1 for patient below 100 kg and 1500 mg for patients above 100 kg, followed by 340 mg SC on Days 2, 8, 15, and 22.

IVIg regimen: 400 mg/kg daily on Day 1, 2, 3, 4 and 5

Each panel corresponds to a different output of the model, i.e. Total Crova=[Ab1]+[Ab1Ag]+[AgAb1Ag], total C5=[Ag]+[Ab1Ag]+2[AgAb1Ag], Total IgG=[IgG], Crova free paratopes=2[Ab1]+[Ab1Ag], Free crova=[Ab1], Free C5=[Ag]. All concentrations are expressed in uM.

FIG. 17 Sensitivity Analysis #1: [FcRn]total concentration divided by a factor of 2. Simulated Median and Min/Max Time Profiles for 33 Subjects Receiving Only Crovalimab (in black) or Crovalimab with IVIg (in blue). All Concentrations are in uM

Median (continuous lines) and min/max (dotted lines) simulated time profiles for 33 subjects receiving either crovalimab alone (black lines) or crovalimab with IVIg (blue lines);

Crovalimab regimen: 1000 mg IV on Day 1 for patient below 100 kg and 1500 mg for patients above 100 kg, followed by 340 mg SC on Days 2, 8, 15, and 22.

IVIg regimen: 400 mg/kg daily on Day 1, 2, 3, 4 and 5

Each panel corresponds to a different output of the model, i.e. Total Crova-[Ab1]+[Ab1Ag]+[AgAb1Ag], total C5=[Ag]+[Ab1Ag]+2[AgAb1Ag], Total IgG=[IgG], Crova free paratopes=2[Ab1]+[Ab1Ag], Free crova=[Ab1], Free C5=[Ag]. All concentrations are expressed in uM.

FIG. 18 Sensitivity Analysis #2: Ve Multiplied by 10. Simulated Median and Min/Max Time Profiles for 33 Subjects Receiving Only Crovalimab (in black) or Crovalimab with IVIg (in blue). All Concentrations are in uM

Median (continuous lines) and min/max (dotted lines) simulated time profiles for 33 subjects receiving either crovalimab alone (black lines) or crovalimab with IVIg (blue lines); Crovalimab regimen: 1000 mg IV on Day 1 for patient below 100 kg and 1500 mg for patients above 100 kg, followed by 340 mg SC on Days 2, 8, 15, and 22.

IVIg regimen: 400 mg/kg daily on Day 1, 2, 3, 4 and 5

Each panel corresponds to a different output of the model, i.e. Total Crova=[Ab1]+[Ab1Ag]+[AgAb1Ag], total C5=[Ag]+[Ab1Ag]+2[AgAb1Ag], Total IgG=[IgG], Crova free paratopes-2[Ab1]+ [Ab1Ag], Free crova=[Ab1], Free C5=[Ag]. All concentrations are expressed in uM.

FIG. 19 Sensitivity Analysis #3: IgG Baseline Multiplied by 2. Simulated Median and Min/Max Time Profiles for 33 Subjects Receiving Only Crovalimab (in black) or Crovalimab with IVIg (in blue). All Concentrations are in uM

Median (continuous lines) and min/max (dotted lines) simulated time profiles for 33 subjects receiving either crovalimab alone (black lines) or crovalimab with IVIg (blue lines);

Crovalimab regimen: 1000 mg IV on Day 1 for patient below 100 kg and 1500 mg for patients above 100 kg, followed by 340 mg SC on Days 2, 8, 15, and 22.

IVIg regimen: 400 mg/kg daily on Day 1, 2, 3, 4 and 5

Each panel corresponds to a different output of the model, i.e. Total Crova=[Ab1]+[Ab1Ag]+[AgAb1Ag], total C5=[Ag]+[Ab1Ag]+2[AgAb1Ag], Total IgG=[IgG], Crova free paratopes=2

[Ab1]+ [Ab1Ag], Free crova=[Ab1], Free C5=[Ag]. All concentrations are expressed in uM.

FIG. 20 Sensitivity Analysis #4: C5 Baseline Multiplied by 2. Simulated Median and Min/Max Time Profiles for 33 Subjects Receiving Only Crovalimab (in black) or Crovalimab with IVIg (in blue). All Concentrations are in uM

Median (continuous lines) and min/max (dotted lines) simulated time profiles for 33 subjects receiving either crovalimab alone (black lines) or crovalimab with IVIg (blue lines);

Crovalimab regimen: 1000 mg IV on Day 1 for patient below 100 kg and 1500 mg for patients above 100 kg, followed by 340 mg SC on Days 2, 8, 15, and 22.

IVIg regimen: 400 mg/kg daily on Day 1, 2, 3, 4 and 5

Each panel corresponds to a different output of the model, i.e. Total Crova=[Ab1]+[Ab1Ag]+[AgAb1Ag], total C5=[Ag]+[Ab1Ag]+2[AgAb1Ag], Total IgG=[IgG], Crova free paratopes=2

[Ab1]+ [Ab1Ag], Free crova=[Ab1], Free C5=[Ag]. All concentrations are expressed in uM.

FIG. 21 Simulated Median and Min/Max Time Profiles for 33 Subjects Receiving Only Crovalimab (in black) or Crovalimab with IVIg (in blue). All Concentrations Are in uM

Median (continuous lines) and min/max (dotted lines) simulated time profiles for 33 subjects receiving either crovalimab alone (black lines) or crovalimab with IVIg (blue lines);

Crovalimab regimen: 1000 mg IV on Day 1 for patient below 100 kg and 1500 mg for patients above 100 kg, followed by 340 mg SC on Days 2, 8, 15, and 22.

IVIg regimen: 400 mg/kg daily on Day 1, 2, 3, 4 and 5

Each panel corresponds to a different output of the model, i.e. Total Crova=[Ab1]+[Ab1Ag]+[AgAb1Ag], total C5=[Ag]+[Ab1Ag]+2[AgAb1Ag], Total IgG=[IgG], Crova free paratopes=2

[Ab1]+ [Ab1Ag], Free crova=[Ab1], Free C5=[Ag], Endo: Free IgG=[IgG]e, Endo: Free Crova=[Ab1]e, Endo: Crova-FcRn=[Ab1-FcRn]e, Endo: Free IgG=[IgG]e, Endo: IgG-FcRn=[IgG-FcRn]e. All concentrations are expressed in uM.

FIG. 22 Simulated Individual, Median and Min/Max Time Profiles for 33 Subjects Receiving Only Crovalimab. All Concentrations Are in uM

Individual (continuous thick violet lines), median (continuous thick black lines) and min/max (dotted lines) simulated time profiles for 33 subjects receiving crovalimab alone; Crovalimab regimen: 1000 mg IV on Day 1 for patient below 100 kg and 1500 mg for patients above 100 kg, followed by 340 mg SC on Days 2, 8, 15, and 22.

Each panel corresponds to a different output of the model, i.e. Total Crova=[Ab1]+[Ab1Ag]+[AgAb1Ag], total C5-[Ag]+[Ab1Ag]+2[AgAb1Ag], Total IgG=[IgG], Crova free paratopes=2

[Ab1]+[Ab1Ag], Free crova=[Ab1], Free C5=[Ag]. All concentrations are expressed in uM.

FIG. 23 Simulated Individual, Median and Min/Max Time Profiles for 33 Subjects Receiving Crovalimab and IVIg. All Concentrations Are in uM

Individual (continuous thick violet lines), median (continuous thick black lines) and min/max (dotted lines) simulated time profiles for 33 subjects receiving crovalimab and IVIg;

Crovalimab regimen: 1000 mg IV on Day 1 for patient below 100 kg and 1500 mg for patients above 100 kg, followed by 340 mg SC on Days 2, 8, 15, and 22.

IVIg regimen: 400 mg/kg daily on Day 1, 2, 3, 4 and 5

Each panel corresponds to a different output of the model, i.e. Total Crova=[Ab1]+[Ab1Ag]+[AgAb1Ag], total C5=[Ag]+[Ab1Ag]+2[AgAb1Ag], Total IgG=[IgG], Crova free paratopes=2

[Ab1]+[Ab1Ag], Free crova=[Ab1], Free C5=[Ag]. All concentrations are expressed in uM.

### EXAMPLES

The following Examples illustrate the invention

### Example 1: Antibody and Clinical Trial

### 1.1 Crovalimab

The sequences of the anti-C5 antibody Crovalimab are shown in SEQ ID NO: 1 (heavy chain) and SEQ ID NO: 2 (light chain). Further, the generation of the anti-C5 antibody Crovalimab used in the present invention is described in WO 2016/098356. Briefly, the genes encoding the heavy chain variable domain (VH) of 305LO15 (SEQ ID NO: 3)) were combined with the genes encoding a modified human IgG1 heavy chain constant domain (CH) variant SG115 (SEQ ID NO: 4). The genes encoding the light chain variable domain (VL) of 305LO15 (SEQ ID NO: 5) were combined with the genes encoding a human light chain constant domain (CL) (SK1, SEQ ID NO: 6). Antibodies were expressed in HEK293 cells co-transfected with the combination of heavy and light chain expression vectors, and were purified by protein.

### 1.2 Clinical Trial

BN43118 is a Phase III, randomized, double-blind, placebo-controlled, multicenter clinical study which will evaluate the efficacy, safety, pharmacokinetics (PK) and pharmacodynamics (PD) of crovalimab compared with placebo as add-on therapy to standard of care (SOC) for the treatment of patients with Guillain-Barré syndrome (GBS). This study will enroll approximately 136 participants randomized to double-blinded treatment in a 1:1 ratio to receive either crovalimab or placebo in addition to background therapy. Blinded study drug (crovalimab or placebo) will be administered intravenously at day 1, and subcutaneously at days 2, 8, 15 and 22 in addition to SOC (IVIg). The patients who received background therapy prior to study day 1 must be able to receive the first dose of blinded study drug before the last dose of background therapy. The study will be composed of 4 periods: 5-day screening period, 4-week treatment period, 24-week post-treatment follow up. As the half-life of IVIg and crovalimab are dependent on the recycling by FcRn receptors in the endosome [9], a mathematical model was built to describe the impact of the co-administration of IVIg on crovalimab PK taking into account the binding competition of both molecules to the FcRn receptors. This model is an extension of the crovalimab PK/PD model describing the binding of crovalimab to C5 developed for PNH naïve patients and reported in [10] with the addition of the FcRn competition between IVIg and crovalimab in the endosome. The present report details the different components and assumptions of this model as well as the calibration based on crovalimab COMPOSER clinical data [11] and literature data on the pharmacokinetics (PK) of IVIg. Simulations were performed to measure the impact of IVIg infusion on the PK/PD of crovalimab and to assess if complete C5 inhibition can be maintained over a 28-day period.

### Example 2: Data Used for Modeling

To calibrate the combined IVIg PK and crovalimab PK/PD model, three sets of data were pooled:
Crovalimab COMPOSER clinical study PK/PD data: individual patient's data were used to estimate crovalimab PK as well as to estimate the binding parameters of crovalimab to C5
IVIg PK data: radiolabeled PK time course profiles for IVIg [12] were used to estimate IVIg PK parameters in serum and in the endosome
M281 PK/PD data: the PK time course of the monoclonal anti-FcRn

antibody M281 [13] and its impact on endogenous IgG were used to estimate the volume of endosome and the concentration of FcRn receptors.

Crovalimab COMPOSER data, IVIg PK data and M281 PK/PD data are described in

Example 2.1, Example 2.2 and Example 2.3, respectively. Values for the binding of crovalimab, IVIg and M281 with FcRn were required to establish the model and were fixed to their in vitro values measured by Surface Plasmon Resonance (SPR) as detailed in Example 2.4. Finally, baseline value for IgG was fixed to values representing average level reported in the literature and described in Example 2.5. The data checking and assembly process is identical to the one described in [10].

### 2.1 COMPOSER Study BP39144

COMPOSER is a first-in-human study consisting of four sequential parts, designed to evaluate the safety, tolerability, PK and PD of crovalimab in healthy volunteers (HVs; Part 1) and the safety, tolerability, PK, PD and efficacy of crovalimab in PNH patients naïve to eculizumab (Parts 2 and 4) and PNH patients switching from eculizumab to crovalimab (Parts 3 and 4). Further details on the study designs, dose and regimen used and samples collected are given in [10]. HV and naïve PNH patient (not previously treated with eculizumab) data available in the clinical database at the cut-off date of Jan. 29, 2020, were included in this analysis. Of note, data from patients switching from eculizumab to crovalimab (Parts 3 and 4) were not used in this analysis as GBS patients enrolled in the BN43118 study will be C5 inhibitor treatment naïve [10]. The concentration of crovalimab, total C5 and free C5 were converted in molar unit (i.e. nM) using the molecular weights of 190 kDa, 149 kDa respectively for C5 and crovalimab. The dose amount and dose rate were normalized by each subject body weight at baseline and converted in mole units (i.e. amounts are in nmol/kg and infusion rates for the IV administration are in nmol/day/kg).

The number of samples available for analysis from the COMPOSER trial for total crovalimab, free and total C5 in serum are given on Table 1.

TABLE 1
Number of Samples Available for Modeling from COMPOSER

| | Part 1 | Part 2 | Part 4* |
|---|---|---|---|
| Variable | (n = 15) | (n = 10) | (n = 8) |
| Total | 149 | 365 | 113 |
| Crovalimab | | | |
| Total C5 | 248 | 315 | 113 |
| Free C5 | 248 | 315 | 110 |

*Only eculizumab naïve patients from COMPOSER part 4 are included in the analysis

### 2.2 IVIG PK Time Course Profiles

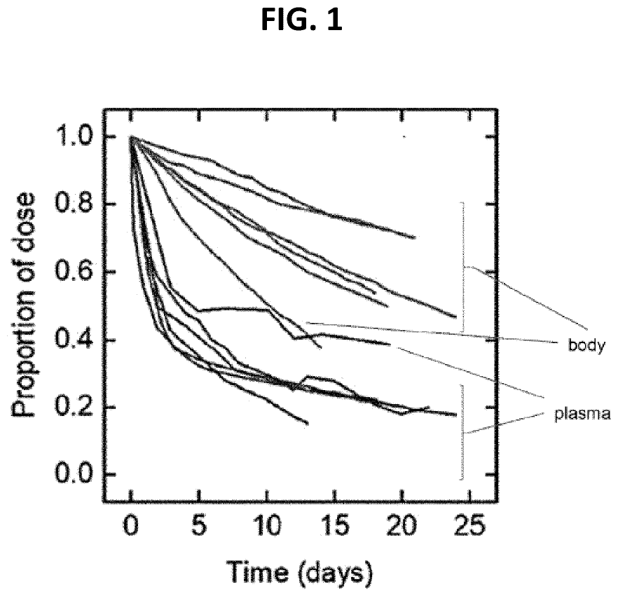

The IVIg PK time profiles of six individuals, displayed on FIG. 1, were extracted from the literature on IVIg radiolabeled studies by Kendrik et al. [12]. The data for an individual subject consist of the time course of the proportion of injected dose of IVIg remaining in serum and the time course of the proportion of dose remaining in the body. Several individuals have health conditions which may result in different PK half-lives and an increased or decreased serum IgG concentration levels. However, subject health status was not taken into account in this analysis.

### 2.3 M281 PK/PD Time-Course Profiles

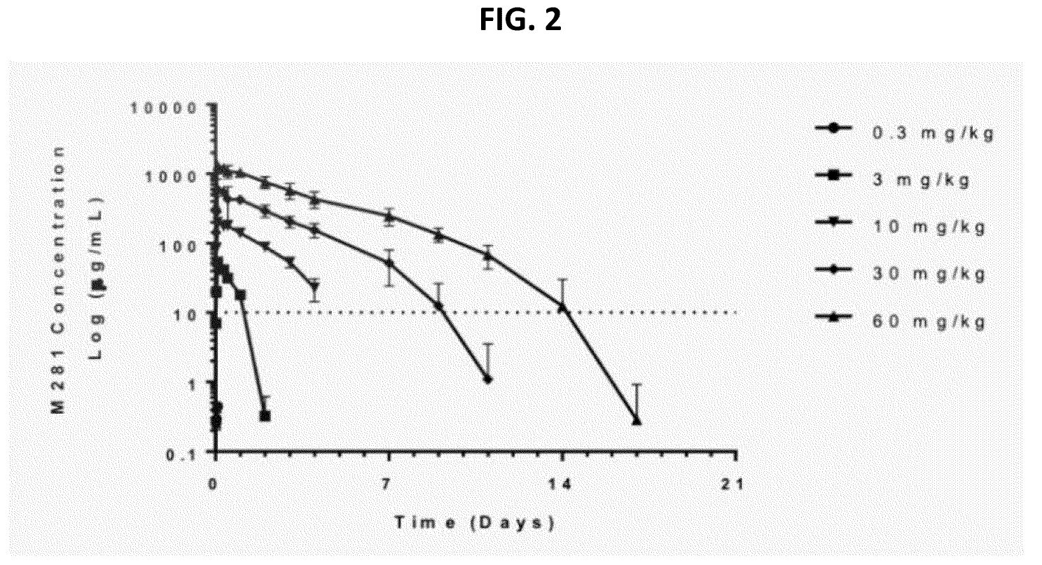

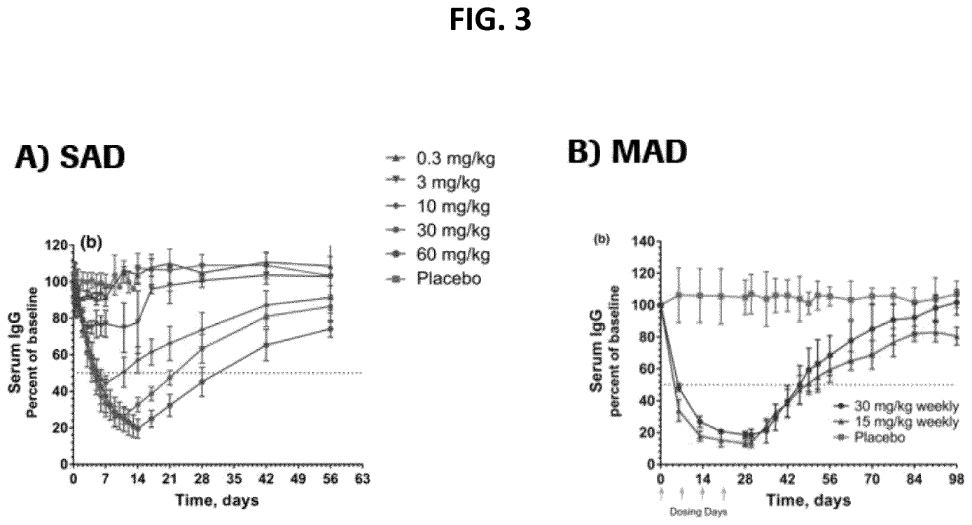

To quantify the volume of the endosome and the number of FcRn receptors available in the endosome, data from the M281 monoclonal IgG1 anti-FcRn antibody [13], a high-affinity FcRn binder (Kd=28.7 pM at pH=6.0), were used for model calibration. Average PK profiles from First-in-Human (FIH) Single Ascending Dose (SAD) study in healthy volunteers for 3 mg/kg (n=3), 10 mg/kg (n=6), 30 mg/kg (n=6) and 60 mg/kg (n=6) doses were extracted from FIG. 2 (reported in [14]). As the PK profiles in the M281 MAD study were not available from the literature, only the SAD study informed the PK of IgG. As M281 has high-affinity to FcRn at endosomal pH=6.0, it blocks binding of endogenous IgG to FcRn and decreases serum IgG concentration. FIG. 3 displays the reduction of the average serum endogenous IgG profiles for increasing doses of M281 in the SAD and MAD studies. These data provides information on the average level of FcRn receptors available for IgG recycling in the endosome. The average serum IgG concentrations (as percentage relative to baseline) were extracted from this figure for the SAD doses of 3 mg/kg (n=3), 10 mg/kg (n=6), 30 mg/kg (n=6) and 60 mg/kg (n=5) and for the MAD doses of 15 mg/kg (n=3), 30 mg/kg (n=3).

We note that the MAD time profiles for IgG were used in the model calibration even though the PK profiles in the M281 MAD study were not available. As individual subjects' data were not available for M281, we assumed that each average profile for the different dose arms in the SAD and MAD studies corresponds to a different “individual” in the dataset used for model calibration (and which comprises the individual data for crovalimab and the individual radiolabeled IgG PK profiles).

### 2.4 Crovalimab, IVIG and M281 FcRn Binding

To model the interactions of Crovalimab, IgG and M281 with the FcRn receptors in the endosome, the in vitro association rate constants kon and the dissociation rate constant koff between Crovalimab, IgG, M281 and FcRn were obtained from the literature and reported in Table 2.

TABLE 2
FcRn binding rates and constants at pH = 6.0

| | kon | koff | Kd (nM) = |
|---|---|---|---|
| Molecules | (nM−1day−1) | (day−1) | koff/kon |
| Crovalimab | 1.35 | 230 | 170 |
| IgG | 1 | 100 | 100 |
| M281 | 141 | 3.91 | 0.0287 |

Kd Crovalimab data from [7]; koff Crovalimab set to the IgG value reported in [16]; IgG data from [12]; M281 data are from [13].

### 2.5 IgG Baseline Concentration

As the endogenous baseline IgG concentration was not available in any of the dataset, we assume for model calibration and simulation that every patient had the same baseline IgG concentration. This concentration was fixed to 1 g/dL (i.e. 66.7 uM assuming a molecular weight of 150e3 g/mol for IgG) as a normal IgG concentration range in adults has been reported [17] to be between 0.767 g/dL and 1.59 g/dL.

### Example 3: Modeling

### 3.1 Methodology

### a) Model Calibration Strategy

A pooled dataset consisted of Crovalimab COMPOSER data, IVIg PK data and M281 PK/PD data (and described in Example 2) was used for the simultaneous calibration of the crovalimab PK/PD, IVIg PK and M281 PK/PD model. Population parameters estimates were obtained using a Non-Linear Mixed Effect (NLME) approach and individual parameter estimates were derived using empirical Bayes estimates (EBEs) as described in [10].

### 3b) Software

The non-linear mixed effect analyses were performed using the Monolix software system, version 2019R2 (Lixoft, Paris, France) to obtain parameter estimates. Simulations were performed with the R package mIxR version 4.1.5 in the R environment version 3.6.3.

### c) Patient Data Inclusion Criteria

All available subject/patient data from COMPOSER Part 1, 2 and 4 described in Table 1 are included in this analysis.

### d) Notations

In the following part of the document, compact notations defined in Table 3 are used to represent serum concentration for crovalimab, C5, endogenous IgG or IVIg, radiolabeled IVIg, M281 and the complexes formed by the binding of crovalimab with C5. Some of these quantities are also estimated in peripheral compartment (with the suffix p) and in the endosome (with the suffix e).

TABLE 3
Notations Used to Describe Free Molecules, Complexes
in Serum, Peripheral Compartment and Endosome

| | | Concentration | Concentration | |
|---|---|---|---|---|
| | Concentration | in peripheral | in the | |
| Molecule | in serum | cmpt. | endosome | Interpretation |
| Ab1 | [Ab1] | [Ab1]p | [Ab1]e | Free |
| | | | | crovalimab |
| Ag | [Ag] | | | Free C5 in |
| | | | | serum |
| Ab1Ag | [Ab1Ag] | [Ab1Ag]p | | Crovalimab |
| | | | | bound to one |
| | | | | C5 |
| AgAb1Ag | [AgAb1Ag] | [AgAb1Ag]p | | Crovalimab |
| | | | | bound to two |
| | | | | C5 |
| Ab1 free | 2 [Ab1] + | | | Ab1 free |
| paratopes | [Ab1Ag] | | | paratopes |
| IgG | [IgG] | [IgG]p | [IgG]e | Endogenous |
| | | | | IgG and IVIg |
| IgG* | [IgG*] | [IgG*]p | [IgG*]e | Radiolabeled |
| | | | | IVIg |
| M281 | [M281] | | [M281]e | M281 |
| | | | | concentration |
| FcRn | | | [FcRn]e | Free FcRn |
| FcRn − Ab1 | | | [FcRn − Ab1]e | FcRn bound to |
| | | | | Ab1 |
| FcRn − IgG | | | [FcRn − IgG]e | FcRn bound to |
| | | | | IgG |
| FcRn − IgG* | | | [FcRn − IgG*]e | FcRn bound to |
| | | | | radiolabeled |
| IgG | | | | |
| FcRn − M281 [ | | | FcRn − IgG*]e | FcRn bound to |
| | | | | M281 |
| | [Ab1]total | | | Total Ab1 |
| | | | | concentration |
| | [Ag]total | | | Total Ag |
| | | | | concentration |
| | [FcRn]total | | | Total FcRn |
| | | | | concentration |

3.2. Model of Ab1, Ag, IVIg and M281 with FcRn Recycling in the Endosome

### a) Model Description

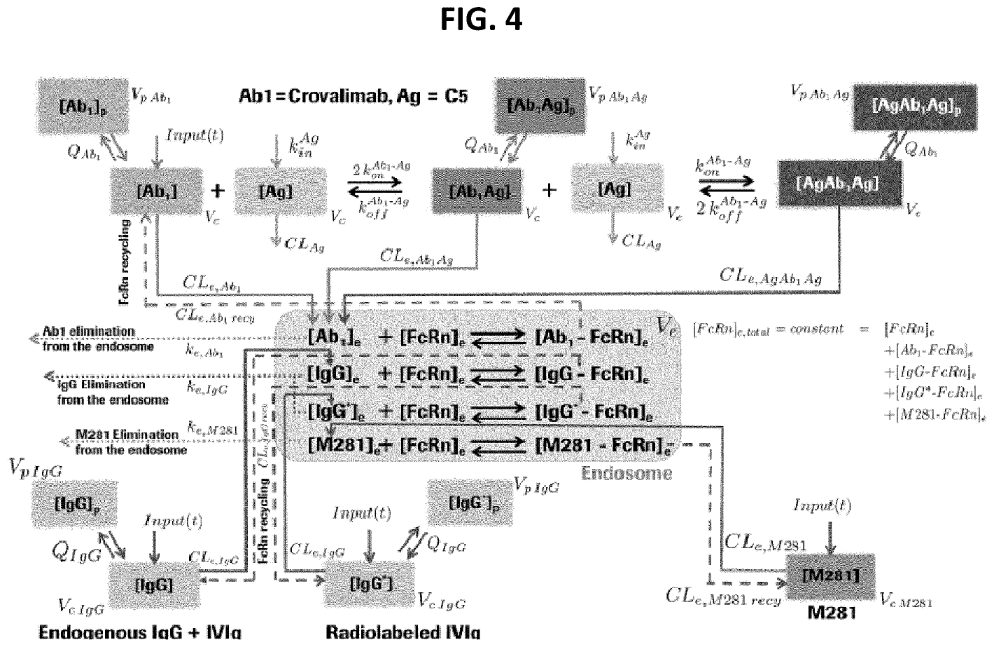

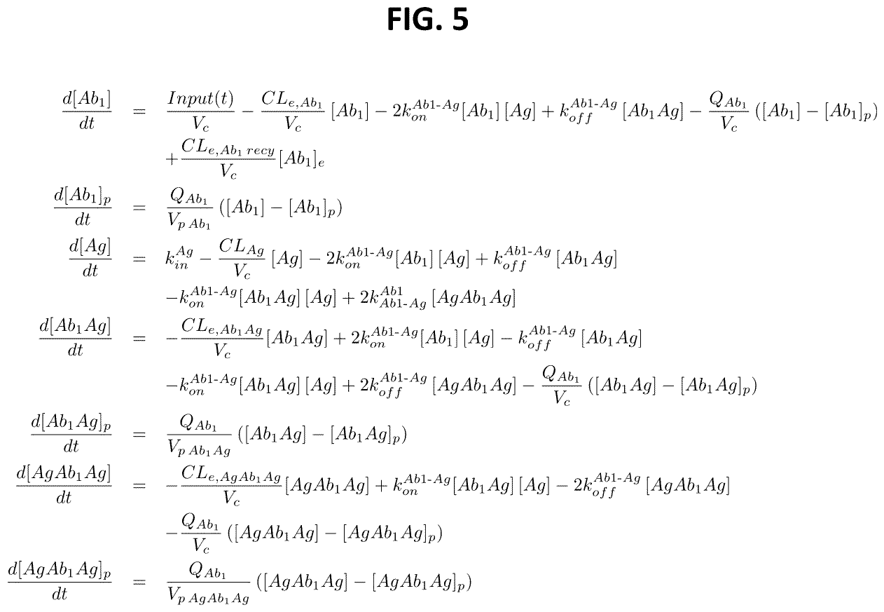

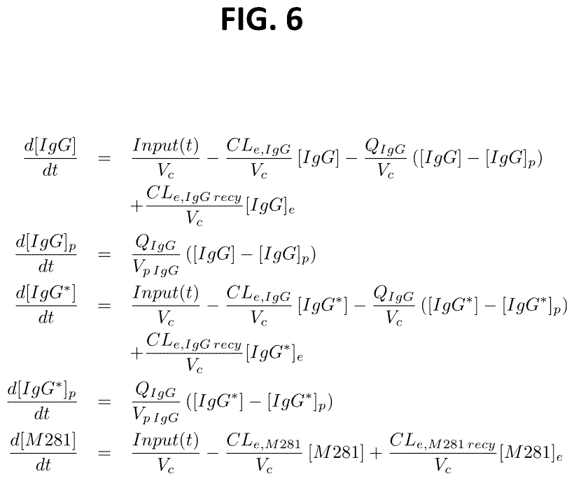

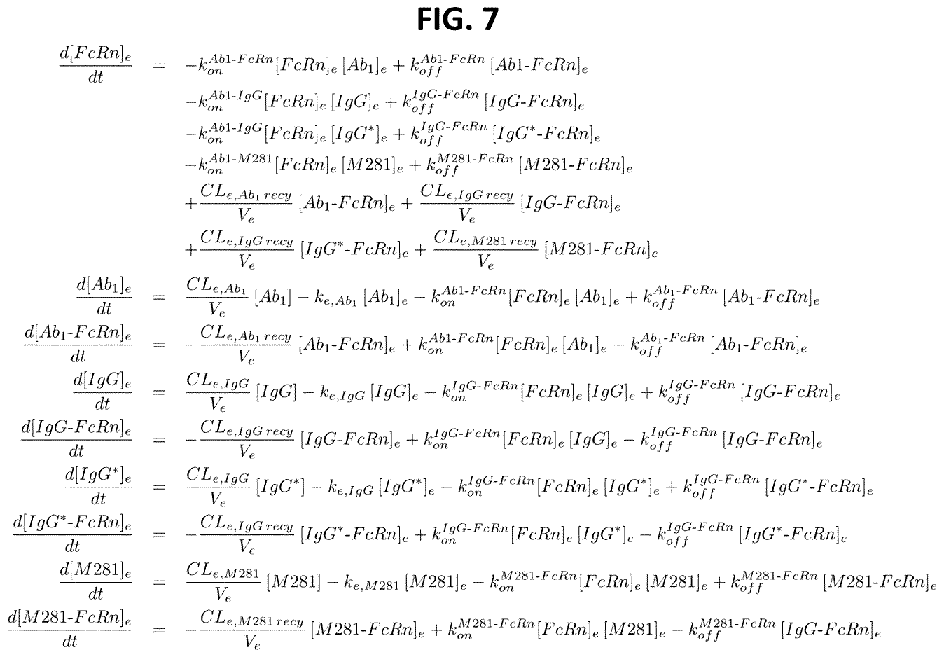

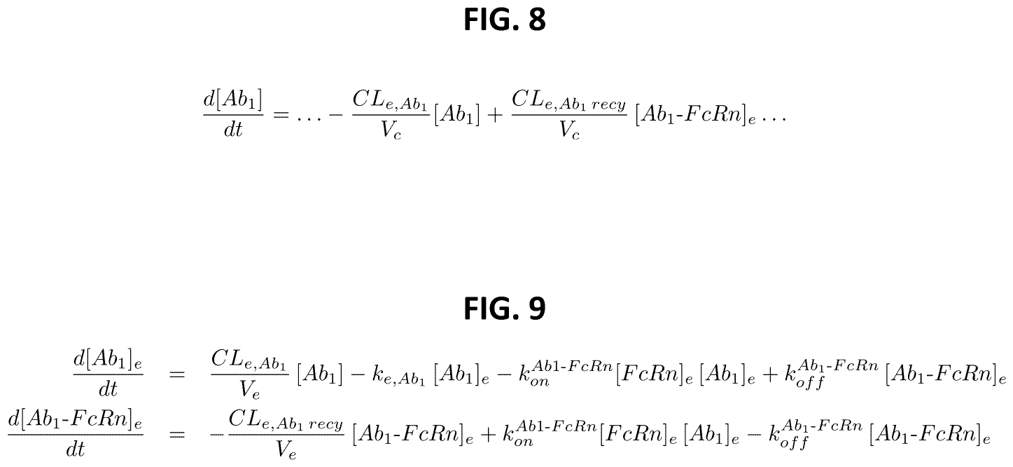

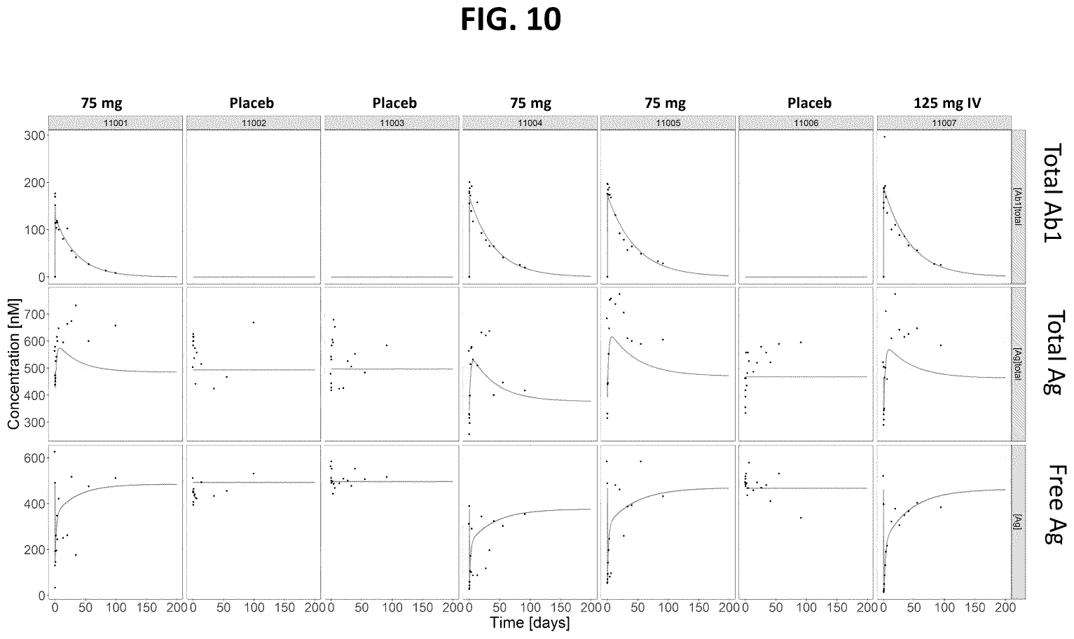

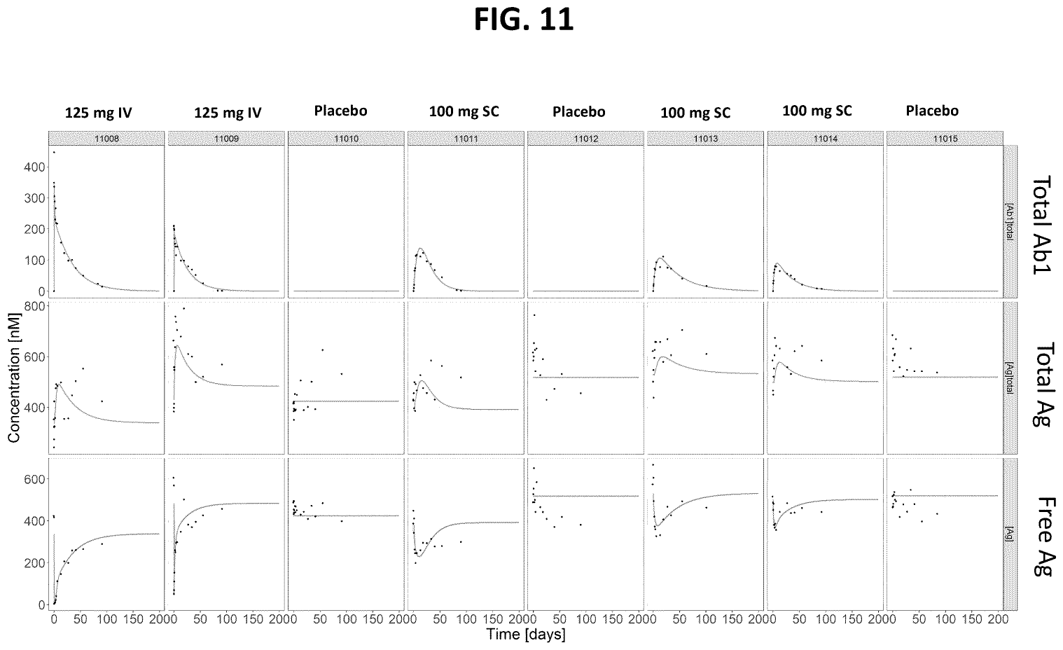

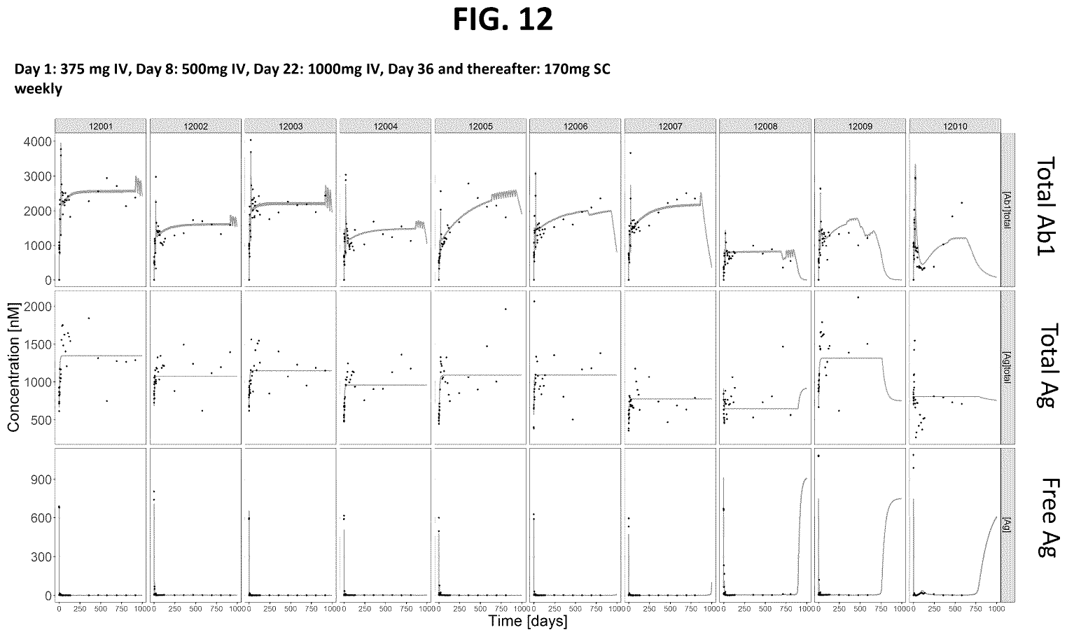

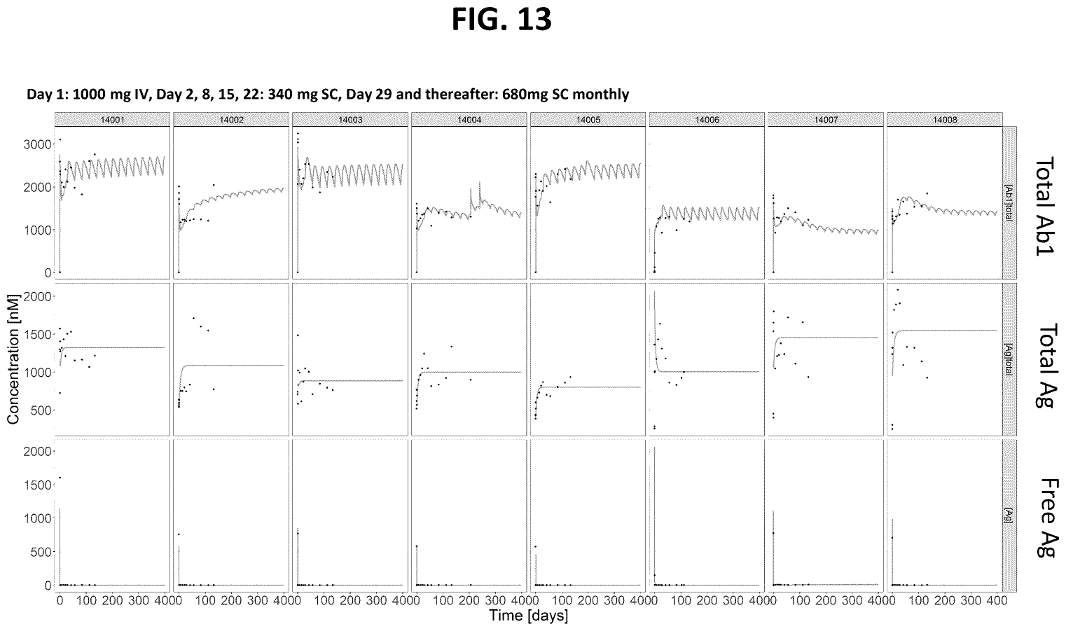

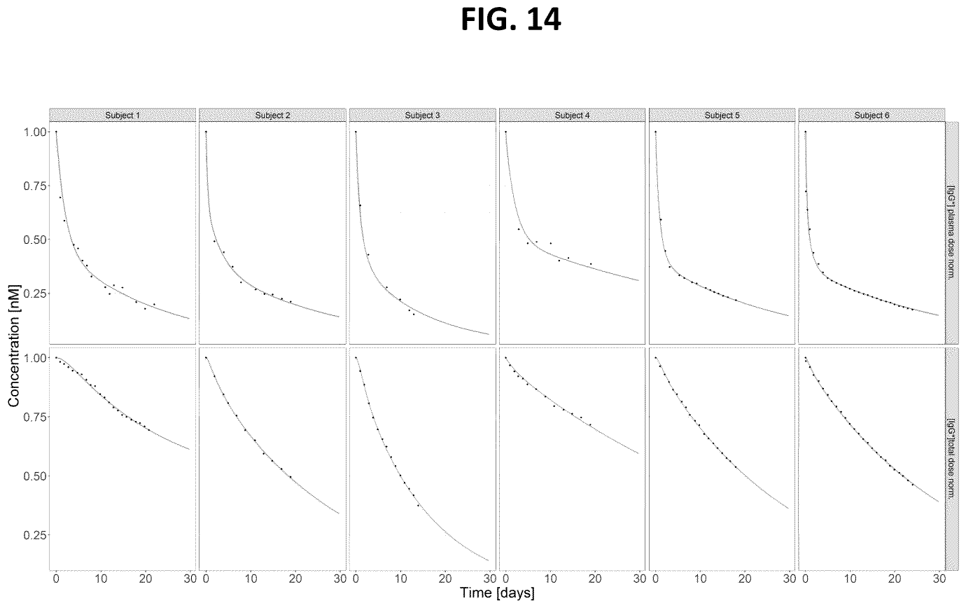

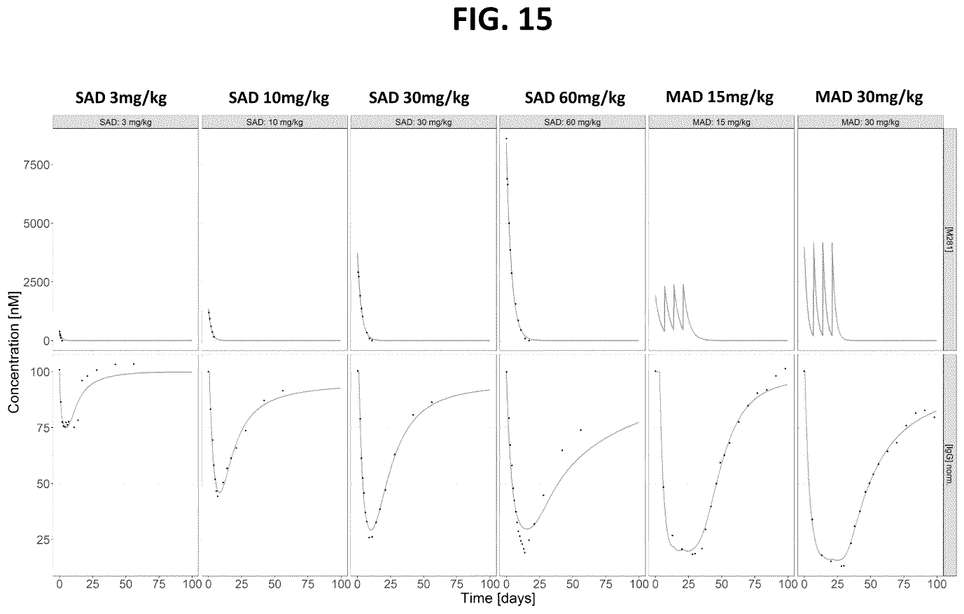

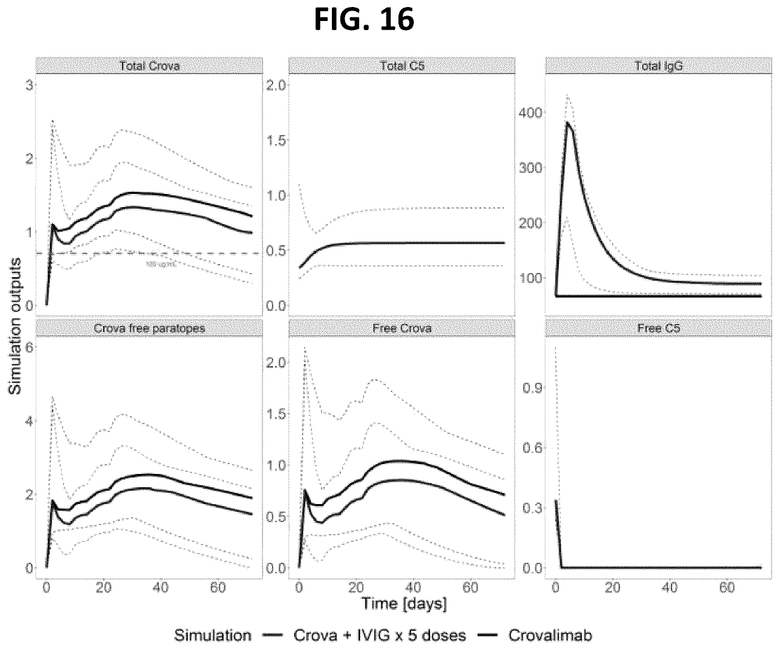

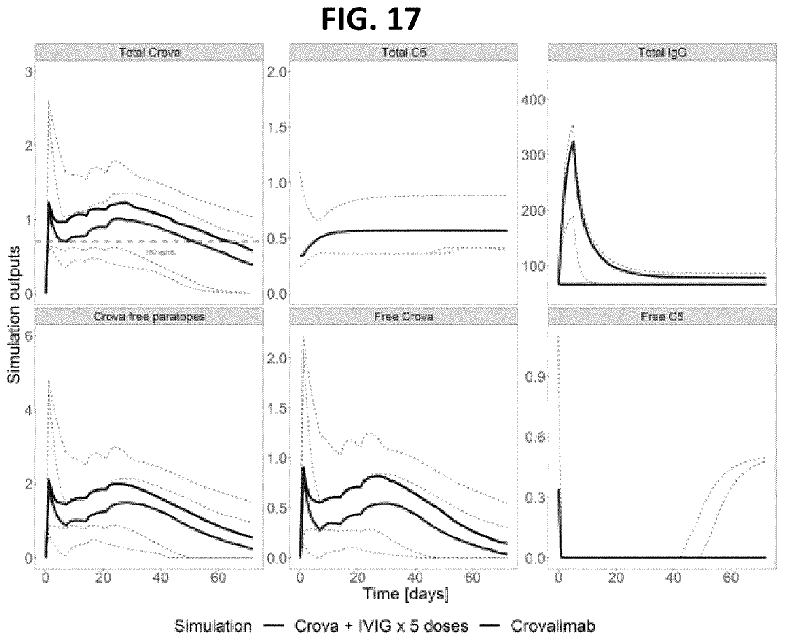

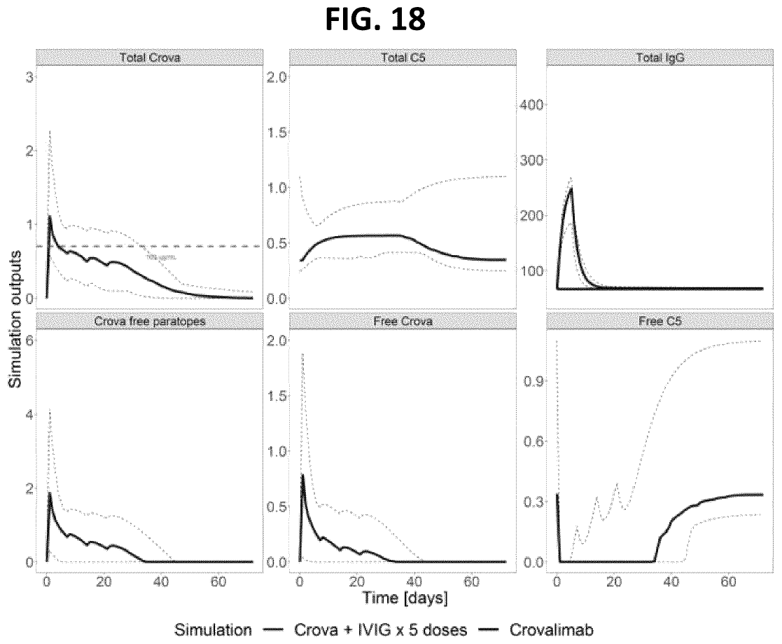

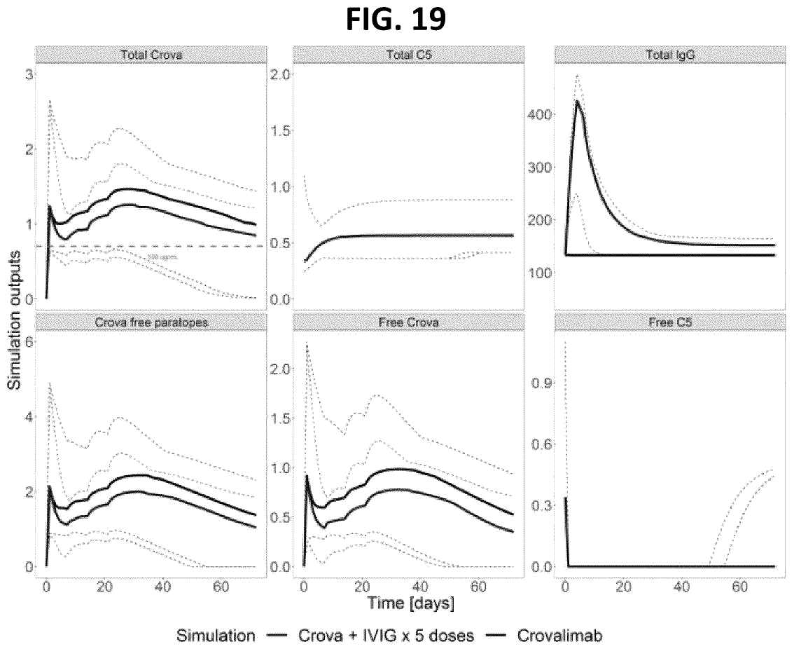

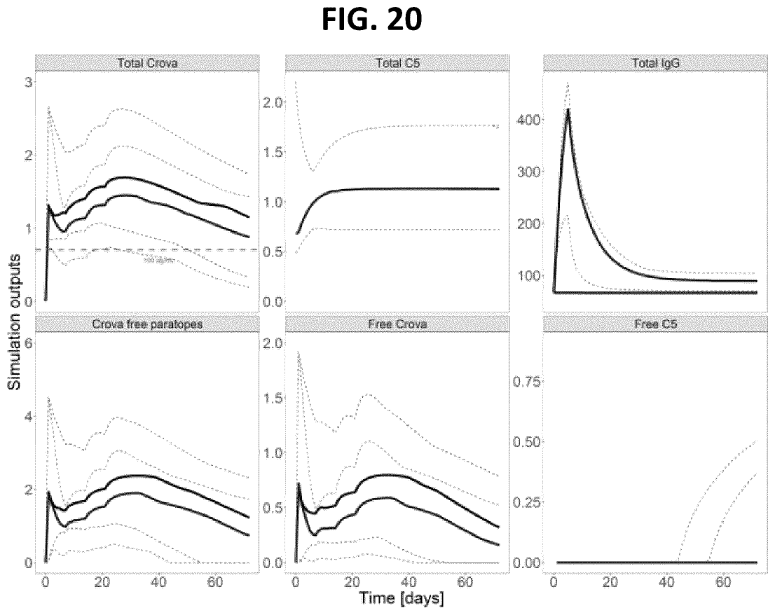

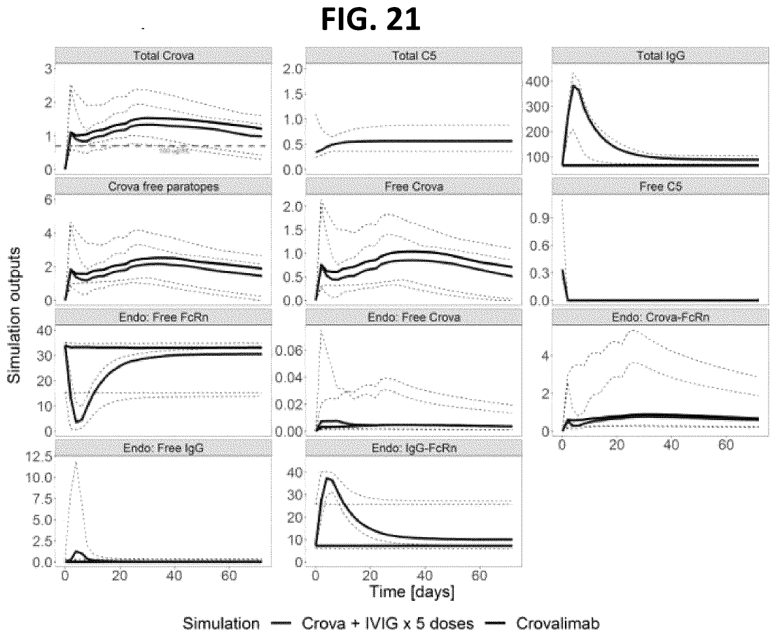

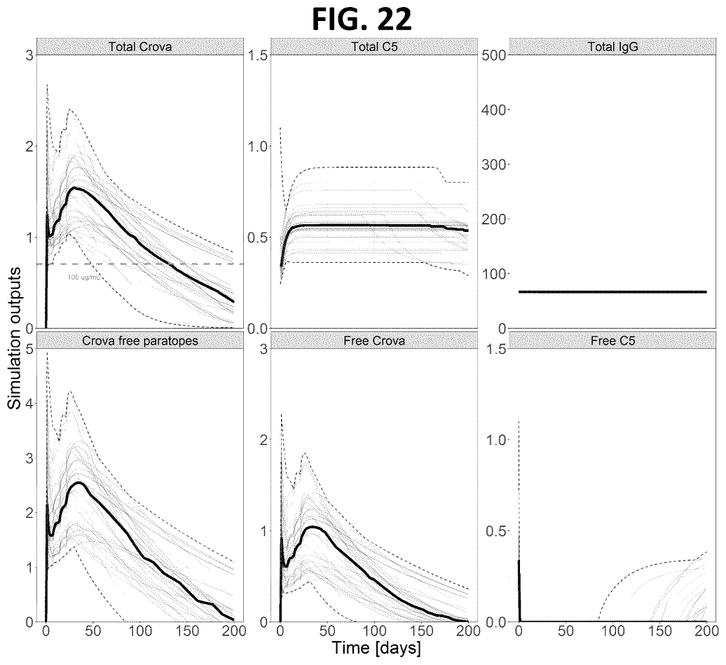

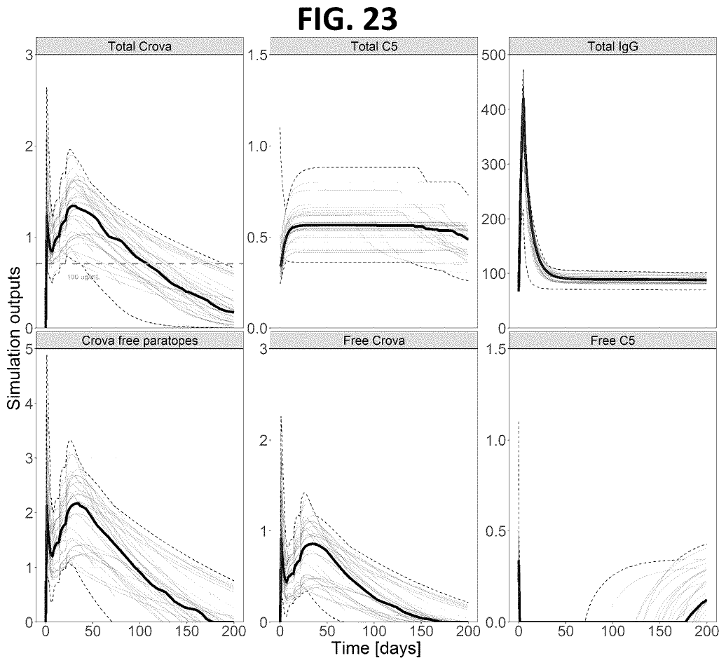

The model describing the binding of Ab1 (crovalimab) with Ag (C5) and the competition with IgG for FcRn recycling in the endosome is displayed on FIG. 4. It comprises the following components:
Ab1 PK/PD model in serum: the upper part of FIG. 4 contains two binding sub-models describing the sequential binding process of one Ag protein to one arm of the free Ab1 antibody to form the complex Ab1Ag, followed by a second Ag protein binding the second arm of the same antibody to form the complex AgAb1Ag (as described in [10]). Peripheral distribution compartments were included for free and bound Ab1 with two different volumes of distribution for free Ab1 and the complexes Ab1Ag and Ag Ab1Ag. During the modeling process, it was observed that the data did not support the addition of a peripheral compartment for Ag. The Ordinary Differential Equations (ODEs) describing the concentrations of free Ab1 (i.e. [Ab1]), free Ag (i.e. [Ag]) and Ab1 bound to one Ag (i.e. [Ab1Ag]) and two Ag (i.e. [AgAb1Ag]) are given in FIG. 5
   IgG PK model in serum: the lower left part of FIG. 4 is a two-compartment linear disposition model for endogenous IgG or IVIg and radiolabeled IVIg (annotated with a star, i.e. IgG*). The volumes of distribution and the clearances are assumed identical for IgG and IgG*. The ODEs describing the concentrations of IgG and IgG* in serum are given in FIG. 6.
      M281 PK model in serum: a one-compartment linear disposition model is used to described M281 PK. The ODE describing the concentrations of M281 in serum is given in FIG. 6.
      Endosome model: after internalization into the endosome, crovalimab, IgG, IgG* and M281 antibodies can bind to FcRn and being recycled back into serum. The antibodies not bound to FcRn are eliminated from the endosome. Since Ab1 has been designed with pH dependent recycling technology (i.e. SMART-Ig Recycling®), we assume that when the antibody is internalized in the endosome, Ag dissociates from the antibody complexes Ab1Ag and AgAb1Ag due to the thousand-fold increase in the dissociation constant KdAb1 (see [7]). Therefore, only free Ab1 is present in the endosome. The ODEs describing the binding to FcRn in the endosome are given in FIG. 7.

The definition and the description of the model parameters are given in Table 4.

TABLE 4
Definition and Unit of the Model Parameters

| Parameter | Description | Units |
|---|---|---|
| k [symbol] | Ab₁-Ag association rate | nM |
| k [symbol] | Ab₁-Ag dissociation rate | day⁻¹ |
| k [symbol] | Production rate of free Ag | nmol/day |
| Input( [symbol] ) | Dosing input | nmol/kg |
| CL_{Ag} | Clearance rate of free Ag | L/day/kg |
| CL [symbol] | Clearance of free Ab₁ into the endosome | L/day/kg |
| CL [symbol] | Clearance of Ab₁Ag complex into the | L/day/kg |
| | endosome | |
| CL [symbol] | Clearance of AgAb₁Ag complex into | L/day/kg |
| | the endosome | |
| CL [symbol] | Clearance of IgG into the endosome | L/day/kg |
| CL [symbol] | Clearance of M281 into the endosome | L/day/kg |
| V [symbol] | Volume central cmpt. for Ab₁ and Ag | L/kg |
| V [symbol] | Volume central cmpt. for IgG | L/kg |
| V [symbol] | Volume central cmpt. for M281 | L/kg |
| Q_{Ab} [symbol] | Ab₁, Ab₁Ag and AgAb₁Ag | L/day/kg |
| | inter-compartmental clearance | |
| Q [symbol] | IgG inter-compartmental clearance | L/day/kg |
| V [symbol] | Volume peripheral cmpt. for free Ab₁ | L/kg |
| V [symbol] | Volume peripheral cmpt. for Ab₁Ag | L/kg |
| V [symbol] | Volume peripheral cmpt. for AgAb₁Ag | L/kg |
| V [symbol] | Volume peripheral cmpt. for IgG | L/kg |
| V [symbol] | Volume peripheral cmpt. for M281 | L/kg |
| [FcRn]_{e, total} | Total number of FcRn in the endosome | nM |
| V [symbol] | Volume endosome | L/kg |
| k [symbol] | Ab₁-FcRn association rate | nM |
| k [symbol] | Ab₁-FcRn dissociation rate | day⁻¹ |
| k [symbol] | IgG-FcRn association rate | nM |
| k [symbol] | IgG-FcRn dissociation rate | day⁻¹ |
| k [symbol] | M281-FcRn association rate | nM |
| k [symbol] | M281-FcRn dissociation rate | day⁻¹ |
| CL [symbol] | Clearance describing the recycling of Ab₁ into | L/day/kg |
| | the plasma from the complex Ab₁-FcRn in | |
| | the endosome | |
| CL [symbol] | Clearance describing the recycling of IgG into | L/day/kg |
| | the plasma from the complex IgG-FcRn in | |
| | the endosome | |
| CL [symbol] | Clearance describing the recycling of M281 | L/day/kg |
| | into into the plasma from the complex M281- | |
| | FcRn in the endosome | |

[symbol]

indicates data missing or illegible when filed

The binding constant konAb1-Ag and koffAb1-Ag describe the binding of one arm of Ab1 antibody with Ag. Therefore, a free antibody which has two free Fab arms available has a two-times higher probability to bind a free Ag than an antibody where one arm is already bound to one Ag. This is reflected in the model equation as 2konAb1-Ag in the binding equation of free Ab1 on FIG. 5. Similarly, an antibody bound to two Ag, has 2-times higher probability to lose an Ag molecule than an antibody bound to only one Ag. This is reflected in the equation describing the dissociation of two Ag bound to an antibody (i.e. AgAb1Ag) by a factor of 2 on the dissociation rate, i.e. 2 koffAb1 on FIG. 5.

To describe endosome internalization and FcRn recycling for Ab1, a clearance term CLe, Ab1 is added with a negative sign to the ODE equation for Ab1 in serum as shown on FIG. 8. The same term appears with a positive sign (and after adjustment for the different volume of distribution between serum Vc and endosome Ve) in the equation describing free Ab1 in the endosome as displayed in FIG. 9. After binding to FcRn, Ab1 is recycled back to serum through the clearance term CLe, Ab1 recy which appears with a negative sign on the ODE equation for the complex Ab1-FcRn in FIG. 9 in the endosome and with a positive sign in the serum equation for Ab1 in FIG. 8. Ab1 antibodies not bound to FcRn in the endosome are eliminated with the term ke, Ab1 in FIG. 9.

Similar parameters and equations were introduced in the model to describe the internalization and recycling of IgG, IgG* and M281 as shown on FIG. 7.

### b) Summary of Model Assumptions

The main hypotheses of this model are the following:
The binding rates (konAb1-Ag and koffAb1-Ag) of Ag to Ab1 are identical if the second arm of the Ab1 antibody is free or already bound to another Ag protein
The koffAb1 was set to a fixed value for each subject, assuming that in vitro Surface Plasmon Resonance (SPR) estimation of this constant reflects the in-vivo situation
The binding of Ag to Ab1 only occurs in the central compartment (no binding in peripheral tissue)
The production rate (kinAg) and elimination rate (CLAg) of endogenous Ag is constant over time.
Clearance processes to the endosome are linear and not saturable.
Only free Ab1 antibody is recycled (i.e. Ab1Ag and AgAb1Ag are recycled as Ab1) since we assume that in the endosome Ab1 is not bound to Ag due to the pH dependent recycling technology SMART-Ig Recycling®.
The total [FcRn]total concentration is assumed constant over time
FcRn recycling processes from the endosome into the serum are assumed linear and not saturable (after binding to FcRn).
Since albumin binds to a different FcRn epitope than IgG, we consider that albumin is not involved in the FcRn recycling saturation process of IgG antibodies.
Only free Ab1, IgG, IgG* and M281 are cleared from the endosome (i.e. all the molecules bound to FcRn are recycled back to serum).
We assume the same elimination constant in the endosome for Ab1, IgG, IgG* and M281, i.e. ke Ab1=ke IgG=ke M281.
We assume that antibodies removal from the body happens only in the endosome. Thus, no other parallel process is considered which would require the addition of other elimination constants

### Example 4: Simulations and Sensitivity Analysis

### 4.1 Base Case Simulation

Simulations of patients receiving either crovalimab alone or co-administered with IVIg (with both treatment starting on the same day) were performed in using the following regimen:
Crovalimab: 1000 mg IV on Day 1 for patient below 100 kg and 1500 mg for patients above 100 kg, followed by 340 mg SC on Days 2, 8, 15, and 22
IVIg: 400 mg/kg daily on Day 1, 2, 3, 4 and 5.
The simulations were performed using the EBEs obtained from the 33 subjects from the COMPOSER study which were included in the model calibration. The EBEs for the subjects from the radiolabeled IVIg and M281 studies were not used in the simulations since they do not contain any information on C5 inhibition.
For each subject, a C5 baseline concentration of 70 ug/mL and an IgG baseline concentration of 1 g/dL was assumed.
The main outputs of these simulations are the median and min/max time-course profiles values for:
Total Ab1 drug concentrations (in uM)
Total IgG concentrations (in uM)·
Free Ab1 (in uM)·
Free Ab1 paratope concentrations (in uM), i.e. the number of free Ab1 arms available for binding to Ag. This quantity is given by the sum 2×[Ab1]+[Ab1Ag]

In addition, endosome time course profiles for free FcRn, free IgG, free crovalimab and the complexes FcRn-crovalimab, FcRn-IgG are provided in the appendix.

The main metric used to compare the simulations between crovalimab alone and crovalimab with IVIg is the maximum reduction in median PK concentration at trough (at the time of crovalimab administration) and the duration of complete C5 inhibition.

### 4.2 Sensitivity Analysis

To assess the robustness of the base case simulation, sensitivity analyses were performed to lower the threshold to saturate FcRn recycling with the following scenarios:
dividing by 2 the concentration of FcRn receptors in the endosome.
multiplying by 10 the volume of the endosome
multiplying by 2 the baseline IgG concentration

In addition, as GBS is an acute condition, individual variations in C5 levels are unknown; then, a simulation assuming an increase of baseline C5 concentration from 70 ug/mL to 140 ug/mL was performed in this sensitivity analysis.

All the simulations were performed using the same dosing regimen, same set of parameters representing the same 33 patients described in Example 4.1. In addition, the same metric was used to compare the sensitivity analysis with or without IVIg co-administration.

### Example 5: Results

### 5.1 Modeling

The model (see FIG. 4) describing the binding of Ag (i.e. C5) with Ab1 (i.e. crovalimab) and its competition with IgG in the endosome was calibrated using the data from the 15 healthy volunteers in COMPOSER Part 1, 10 and 8 naïve PNH patients from Parts 2 and 4, respectively and the radiolabeled IgG* and M281 data described in Example 2. The number of available samples from COMPOSER Parts 1, 2 and 4 for total Ab1, total Ag, free Ag are given in Table 1.

To reduce the uncertainty on the estimation due to the introduction of the endosome part of the model, the population parameters for the central volume of distribution, the volume and inter-compartment clearance of Ab1 and its complexes with Ag and the Ag production and clearance rates were fixed to the value obtained without the endosome models and described in [10]. These values are reported in Table 5.

The population parameter values obtained from the model calibration are reported on Table 6.

Individual goodness-of-fit plots reported on FIG. 10, FIG. 11, FIG. 12, FIG. 13, show that the concentration time course for total [Ab1], total [Ag] and free [Ag] from COMPOSER study are adequately described by the model.

Individual goodness-of-fit plots on FIG. 14 shows that the model described adequately the dose normalized radiolabeled IgG* in serum on the upper panel and total body radioactivity on the lower panel.

Similarly, FIG. 15 shows that M281 PK and its impact on baseline normalized endogenous IgG concentrations is adequately described by the model for each arm of the SAD and MAD studies.

TABLE 5
Model Population Parameters Estimates Obtained from
the Model Without The Endosome and Reported in [10]
and Fixed in the Endosome Model.

| | | | |
|---|---|---|---|
| | | Median | Inter-Individual |
| Parameter | Unit | (RSE %) | Variability (RSE %) |

Fixed and random effects

| | | | |
|---|---|---|---|
| ka [symbol] | 1/day | 0.114 | CV = 113% |
| kᵢₙ^{Ag} | nM/day | 232 | CV = 22.5% |
| CL_{Ag} | L/day/kg | 0.0166 | CV = 22.8% |
| K_{d}^{Ab1Ag} = | nM | 0.0236 | CV = 25.1% |
| k_{off}^{Ab1Ag}/kₒₙ^{Ab1Ag} | | | |
| K_{off}^{Ab1Ag} | 1/day | 18.8 ^{a} | — |
| Vc | L/kg | 0.0428 | CV = 20.2% |
| Vp_{Ab1} | L/kg | 0.0807 | CV = 55.2% |
| Vp_{Ab1Ag} | L/kg | 0.0065 | CV = 71.1% |
| Q_{Ab1} | L/day/kg | 0.0244 | CV = 157% |

Covariate effect

| | | | |
|---|---|---|---|
| Effect of WT on clearance | — | −0.25^{b} | — |

Model parameters are defined in Table 4, RSE = relative standard error of estimate, CV = coefficient of variation, OFV = objective function value, AIC: Akaike information criterion, BIC: Bayesian information criterion WT = body weight, a = additive error, b = proportional error SD.
The effect of WT on clearance was applied on the following parameters: CL_{Ag} and Q_{Ab1}
^{a} Fixed value without random effect [symbol]
indicates data missing or illegible when filed

TABLE 6
Model Population Parameters Estimates

| | | | |
|---|---|---|---|
| | | Median | Inter-Individual |
| Parameter | Unit | (RSE %) | Variability (RSE %) |

Fixed and random effects

| | | | |
|---|---|---|---|
| CL_{e, Ab1} | L/day/kg | 0.00696 (76%) | CV = 211% (32%) |
| CL_{e, Ab1Ag} = CL_{e, AgAb1Ag} | L/day/kg | 0.0103 (7%) | CV = 28.4% (20%) |
| CL_{e, IgG} | L/day/kg | 0.0079 (100%) | CV = 82% (42%) |
| CL_{e, M281} | L/day/kg | 0.0198 (17%) | CV = 36.3% (36%) |
| Vc_{IgG} | L/kg | 0.0227 (101%) | CV = 102% (31%) |
| Vc_{M281} | L/kg | 0.0503 (5%) | CV = 6.7% (61%) |
| Vp_{IgG} | L/kg | 0.367 (141%) | CV = 424% (26%) |
| Q_{IgG} | L/day/kg | 0.00259 (104%) | CV = 109% (35%) |
| [FcRn]ₜₒₜₐₗ | nM | 4.23e+4 (65%) | CV = 55.3% (113%) |
| Ve | L/kg | 0.00204 (109%) | CV = 213% (26%) |
| CL_{e, Ab1 recy} | L/day/kg | 0.0132 (125%) | CV = 82.5% (229%) |
| CL_{e, IgG recy} | L/day/kg | 0.0725 (120%) | CV = 88.8% (76%) |
| CL_{e, M281 recy} | L/day/kg | 1.15e−115 (NaN) | CV = 9080% (NaN) |
| k_{e Ab1} = k_{e IgG} = k_{e M281} | 1/day | 90.2 (86%) | CV = 150% (38%) |

Covariate effect

| | | | |
|---|---|---|---|
| Effect of WT on the clearances | — | −0.25^{b} | — |

Error model

| | | | |
|---|---|---|---|
| [Ab1]ₜₒₜₐₗ: a (additive) | % | 2.2e−16 (14%) | — |
| [Ab1]ₜₒₜₐₗ: b (proportional) | % | 20.5 (3%) | — |
| [Ag]ₜₒₜₐₗ: b (proportional) | % | 24 (3%) | — |
| [Ag]: a (additive) | % | 0.112 (30%) | — |
| [Ag]: b (proportional) | % | 27.5 (4%) | — |
| [IgG*]ₛₑᵣᵤₘ: a (additive) | % | 2.4e−6 (1.67e+5%) | — |
| [IgG*]ₛₑᵣᵤₘ: b (proportional) | % | 5.9 (24%) | — |
| [IgG*]_{total radioactivity}: a (additive) | % | 0.0028 (78%) | — |
| [IgG*]_{total radioactivity}: b (proportional) | % | 0.52 (57%) | — |
| [M281*]: a (additive) | % | 70.8 (25%) | — |
| [M281*]: b (proportional) | % | 3.5 (47%) | — |
| [IgG] for M281: a (additive) | % | 4.5 (20.5%) | — |
| [IgG] for M281: a (proportional) | % | 5.04e−5 (2.87e+4%) | — |

OFV: 22781,
AIC: 22871,
BIC: 23083,
RUN NAME: RUN1_R2019_27APR20_wM281

Model parameters are defined in Table 4, RSE=relative standard error of estimate, CV=coefficient of variation, OFV=objective function value, AIC: Akaike information criterion, BIC: Bayesian information criterion WT=body weight, a=additive error, b=proportional error SD. The effect of WT on clearance was applied on the following parameters: CLe, Ab1, CLe, Ab1Ag, CLe, AgAb1Ag, CLe, IgG, CLe, M281, Q IgG, CLe, Ab1 recy, CLe, IgG recy, CLe, M281 rec

### Example 6: Simulations and Sensitivity Analysis

### 6.1 Base Case Simulation

Simulations were performed using individual parameter estimates (i.e. the EBEs) of the 33 subjects from the COMPOSER study used in the model calibration.

The results of the simulations for subjects receiving either only crovalimab or crovalimab with IVIg are shown on FIG. 16 where time 0 corresponds to Day 1 when treatments are initiated.

Median and min/max simulation profiles for the concentration of crovalimab and IgG in serum and in the endosome are presented on FIG. 21 in Appendix 1. The 33 individual profiles for subject receiving only crovalimab or both crovalimab and IVIg are depicted on FIG. 22 and FIG. 23, respectively.

A decrease in the serum concentration of crovalimab is observed when co-administered with IVIg at the recommended dose in GBS as it competes for FcRn recycling. Crovalimab median trough concentration decreased by 19% on Day 8 in the presence of IVIg.

The median crovalimab concentration over the simulated time remains above approximately 100 ug/mL threshold concentration, which is the value used as reference to achieve complete C5 activity inhibition. The complete inhibition is confirmed when looking at the expected free C5 profile where complete inhibition is observed. In addition, the minimum value for crovalimab free paratopes remains always above 0 indicating that there is always crovalimab binding reserve to capture free C5 molecules over a period of 72 days.

### 6.2 Sensitivity Analysis

To assess the robustness of the assumptions made in the model, a sensitivity analysis was performed on the model parameters which have an impact on the efficiency of the FcRn recycling. In addition, as GBS is an acute condition, individual variations in C5 levels are unknown; then, a simulation assuming an increase of baseline C5 concentration was performed.

This analysis represents potential “worst-case scenarios” in terms of reduction in C5 inhibition induced by the changes in crovalimab PK. The following model parameters were purposely modified one-by-one, as follows:
1. The total concentration of FcRn in the endosome [FcRn]total is divided by a factor of 22. the volume of the endosome Ve is multiplied by a factor 10
3. The production rate of endogenous IgG is increased by a factor of 2 resulting in a doubling of the baseline concentration of IgG to a value of 2 g/dl
4. The concentrations of C5 at baseline was increased by a factor of 2 from 70 ug/mL to 140 ug/mL

The results of the sensitivity analysis No. 1 is shown on FIG. 17. The impact of IVIg on the PK of crovalimab is higher when concentration of FcRn in the endosome [FcRn]total is divided by a factor 2; in this scenario, crovalimab median concentrations is reduced by 27% on Day 8 at trough in the presence of IVIg. Of note, in the base case simulation this reduction was around 19%. Over a 40-day period, the median profile remains above the 100 ug/ml threshold and the minimum value of crovalimab free paratopes remains always strictly positive which ensure complete C5 inhibition.

The results of the sensitivity analysis No. 2 on FIG. 18 demonstrate that an increase of the volume of the endosome by a factor 10, changes the PK profile of crovalimab. However, no differences can be observed on crovalimab median concentrations with or without IVIg co-administration.

In the analysis No 3, increasing the baseline endogenous level of IgG by a factor of 2 (from 1 g/dL to 2 g/dL) as shown on FIG. 19 results in a reduction of median crovalimab concentrations by 22% on Day 8; however, the value of crovalimab free paratopes always strictly positive during a 40-day period. Of note, in the base case simulation this reduction was around 19%.

Finally, in the sensitivity analysis No 4, a simulation assuming 2-fold increase of baseline C5 concentration (140 ug/mL) was performed and results are provided on FIG. 20. Crovalimab median trough concentration decreased by 22% on Day 8.

These sensitivity analyses demonstrate that the proposed crovalimab dosing strategy is expected to provide adequate inhibition of C5 for at least a 40-day period based on the median PK profile even when taking into account uncertainty on some key parameters driving the recycling of crovalimab by FcRn.

### Example 7 Discussion

A previously developed model of crovalimab, reported in [10], was extended to account for the saturation of FcRn recycling when IVIgs are co-administered. A key assumption of the model is that antibody elimination happens only in the endosome (as it is usually hypothesized in the literature [9]) when antibodies are not bound to FcRn. Thus, the half-life of the antibody is driven by how much an antibody can bind the FcRn receptors in the endosome and being recycled. Therefore, the concentration of FcRn receptors and the volume of the endosome are key parameters to be estimated to quantify when FcRn recycling will be saturated and will impact crovalimab serum concentration. The addition of the M281 PK/PD profile in our dataset provided the necessary information to estimate these two parameters since M281 binding to FcRn clearly saturates the recycling of IgG from the endosome to serum as shown on FIG. 3. We note that the population estimate [FcRn]total=42.3 uM for the FcRn receptor concentration in the endosome reported in Table 6 is matching the reported value of 41.2 uM (i.e. [FcRn]total=Rtot/v3=14 umol/0.34 L) in Kendrik's publication [12]. However, the estimated volume of the endosome Ve=0.14 L (assuming a body weight of 70 kg) is 2.4 lower than reported in the same publication (i.e. v3-0.34 L) motivating the use of the M281 data to estimate this parameter.

After calibration, the model provided adequate goodness-of-fit for the PK of IVIg on FIG. 14 and the PK/PD of crovalimab on C5 inhibition as displayed on FIG. 10, FIG. 11, FIG. 12 and FIG. 13. However, part of the variability on total [Ag] could not be captured at the individual level as can be seen on the second rows of FIG. 10, FIG. 11, FIG. 12 and FIG. 13. The model does not have the ability to match the variation of total Ag as the production rate of Ag (i.e. kinAg) is assumed constant over time in the model. This is in particular highlighted on of FIG. 10 and FIG. 11 for the 6 heathy volunteers receiving a placebo (i.e. subjects 11002, 11003, 11006, 11010, 11012, 11015) who have a constant predicted total Ag concentration (i.e. the horizontal green lines) while the measured total Ag vary within a 300 nM range.

At the population level, parameters were usually estimated with poor accuracy (i.e. with RSE above 50%), as given in Table 6, due to the limited individual data for IgG and M281. These findings justified why during the modeling building process, models parameters describing the PK/PD of crovalimab and C5 in serum were fixed to the population values given in Table 5 to reduce estimation uncertainty on the parameters describing the FcRn recycling process. The values in Table 5 were obtained with the model not explicitly describing the processes happening in the endosome and given in [10].

In order to not propagate the uncertainty on population parameter estimates, only simulations using individual EBE parameters were performed. These EBEs were obtained for the 33 subjects receiving crovalimab in the COMPOSER clinical study. The EBE parameters obtained for the subjects coming from the radiolabeled IVIg and M281 study were not used in the simulations since they did not carry any information on the PK/PD of crovalimab.

The simulations showed that the selected crovalimab dosing regimen (1000 mg IV on Day 1 for patient below 100 kg and 1500 mg for patients above 100 kg, followed by 340 mg SC on Days 2, 8, 15, and 22) provides a median crovalimab PK profile above the 100 ug/mL reference threshold (for complete C5 inhibition) when co-administered with IVIg for 5 consecutive days at the dose of 400 mg/kg. The maximum effect of the IVIg co-administration happens on Day 8 with a reduction of the median serum crovalimab concentration of 19%. The level of free paratopes remains always strictly positive over the 72-day period even when considering the minimum values of the prediction (which corresponds to a few number of subjects) as can be observed on the individual profiles on FIG. 23.

Sensitivity analysis demonstrated the robustness of the results by assessing the impact of reducing the concentration of FcRn receptors by a factor 2, increasing the volume of the endosome by a factor 10 and increasing the baseline level of IgG by a factor 2. In all these cases, the median crovalimab serum concentration remains above the 100 ug/mL over a period of 40 days, and the minimum value of the free paratopes remains strictly positive over a 40-day period.

An increase in the C5 baseline from 70 ug/mL to 140 ug/mL had a mild impact on the median PK level as the reduction of the median serum crovalimab concentration on Day 8 was increased from 19% to 22%. This increase is driven by the faster endosome internalization of the crovalimab-C5 complexes compared with free crovalimab (i.e. CLe, Ab1=0.00696 L/day/kg and CLe, Ab1Ag=CLe, AgAb1Ag=0.0103 L/day/kg), which results in an overall faster elimination of crovalimab.

Thus, the selected corvalimab dosing regimen is expected to cover the therapeutic objective of maintaining sustained complete C5 inhibition over 28 days despite to co-administration of IVIg.

### Example 8 Summary/Conclusions

The impact of IVIg infusion on the PK and PD profiles of crovalimab in subject simultaneously receiving both treatments was investigated using a mechanistic mathematical model. This PK/PD model describes the binding of crovalimab with C5 and predicts the concentrations over time of free C5 and free crovalimab paratopes (which quantify the reserve of free crovalimab sites available to bind C5 molecules).

The model also includes a PK model of endogenous IgG in serum as well as the competition of IgG and crovalimab to bind the FcRn receptor in the endosome. This part of the model allows quantifying the impact of the recommended therapeutic dose of IVIg on crovalimab PK concentration. The model also includes a description of the M281 anti-FcRn antibody PK and PD used to estimate the concentration of FcRn receptors and the volume of the endosome.

A population approach was used to calibrate simultaneously the crovalimab PK/PD model, the IVIg PK model and the M281 PK/PD model. A pooled dataset comprising crovalimab COMPOSER data in 33 healthy volunteers and naïve PNH patients, radiolabeled IgG data from literature in six subjects and published PK/PD SAD and MAD data for the M281 monoclonal anti-FcRn antibody [13,14] was built and used for model calibration.

Simulations and sensitivity analyses were then conducted to quantify the impact of IVIg co-administration on the PK and PD profiles of crovalimab using the individual parameter estimates obtained from 33 subjects from the clinical study COMPOSER. These data were included in the calibration dataset.

The following dose and dosing regimen for crovalimab and IVIg were used in these simulations:
Crovalimab: 1000 mg IV on Day 1 for patient below 100 kg and 1500 mg for patients above 100 kg, followed by 340 mg SC on Days 2, 8, 15, and 22
IVIg: 400 mg/kg daily on Day 1, 2, 3, 4 and 5

IVIg dose and dosing regimen are based on the standard treatment of acute GBS (400 mg/kg QD for 5 consecutive days).

The main conclusions of this modeling & simulation analyses are:
The predicted maximum reduction of the median crovalimab serum concentration was 19% on Day 8 when receiving simultaneously IVIg.
Crovalimab median serum concentrations remain over 72 days above approximately 100 ug/mL, the reference threshold expected to provide complete C5 activity. This period is covering the expected targeted therapeutic duration for the BN43118 study of 28 days.
The level of free paratopes remains always strictly positive indicating that there is always a crovalimab binding reserve available.
Sensitivity analysis demonstrated that complete C5 inhibition is maintained over a 40-day period even when model parameters such as the concentration of FcRn receptors, baseline level of IgG and C5 and the volume of the endosome are changed in the direction of reducing the recycling of crovalimab by FcRn.

Overall, according to simulation results and sensitivity analysis the selected crovalimab dosing regimen provides complete C5 inhibition for at least for 40 days.

In summary, the modeling approach presented provides a tool to understand the interaction of IVIg and crovalimab on FcRn recycling and to quantify through simulations the impact of IVIg on crovalimab PK level, crovalimab free binding sites and free C5.

According to this study results, the following dosing regimen is selected to be tested in the clinical study BN43118.
Crovalimab: 1000 mg IV on Day 1 for patient below 100 kg and 1500 mg for patients above 100 kg, followed by 340 mg SC on Days 2, 8, 15, and 22

However, to ensure that the selected dose is adequate to achieve complete C5 inhibition, a dose confirmation step is planned in the study BN43118; actual PK data from 10 GBS subjects will be assessed to confirm the adequacy of the selected dose.

GLOSSARY OF ABBREVIATIONS

| | |
|---|---|
| Ab1 | Crovalimab |
| Ag | C5 |
| AIC | Akaike information criterion |
| AUC | Area under the serum concentration-time curve |
| BIC | Bayesian information criterion |
| BLQ | Below the limit of quantification |
| BW | Body weight |
| C5 | Complement component 5 |
| Cavg | Caverage |
| CCOD | Clinical cutoff date |
| CL | Clearance |
| Cmax | Maximum concentration |
| Ctrough | Trough/minimum concentration |
| CV | Coefficient of variation |
| DV | Dependent variable (observed concentration) |
| EBE | Empirical Bayes estimate |
| FcRn | Neonatal Fc receptor |
| GBS | Guillain-Barré Syndrome |
| GPI-APs | Glycosylphosphatidylinositol (GPI)-anchored proteins |
| IPRED | Individual predicted value |
| IVIg, IVIG | Intravenous immunoglobulin |
| IWRES | Individual weighted residual |
| ka | Absorption constant |
| kg | Kilogram |
| L | Liter |
| LIA | Liposome Immunoassay |
| LOESS | Locally weighted scatterplot smoothing |
| LOQ | Limit of quantification |
| LRT | Log likelihood ratio test |
| μ | Micro |
| M | Molar |
| MAC | Membrane attack complex |
| MW | Molecular Weight |
| m2 | Square meter |
| mg | Milligram |
| min | Minutes |
| mL | Milliliter |
| mol | Mole |
| n | Nano |
| No | Number |
| NLME | Nonlinear Mixed-Effect Model |
| ODE | Ordinary Differential Equation |
| OFV | Objective function value |
| PD | Pharmacodynamics |
| pl | Isoelectric point |
| PK | Pharmacokinetics |
| PK/PD | Pharmacokinetics/Pharmacodynamics |
| PNH | Paroxysmal Nocturnal Hemoglobinuria |
| PRED | Population Predicted value |
| Q | Inter-compartmental clearance |
| RSE | Relative standard error |
| SAE | Serious adverse events |
| SD | Standard deviation |
| SDTM | Study data tabulation model |
| SMART-Ig | Sequential Monoclonal Antibody Recycling Technology |
| SPR | Surface Plasmon Resonance |
| ss | Steady-state |
| t½ | Half-life |
| U | Units |
| UA | Unable to assess |
| V | Volume of distribution |
| VPC | Visual posterior predictive check |
| WRES | Weighted residuals |
| WT | Weight |

SEQUENCES:

| SEQ | | |
|---|---|---|
| ID | | |
| NO: | Description | Sequence |
| 1 | Crovalimab | QVQLVESGGGLVQPGRSLRLSCAASGFTVHSSYYMAWVRQ |
| | heavy chain | APGKGLEWVGAIFTGSGAEYKAEWAKGRVTISKDTSKNQV |
| | | VLTMTNMDPVDTATYYCASDAGYDYPTHAMHYWGQGTLVT |
| | | VSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSCDKTHTCPPCPAPEL |
| | | RRGPKVFLFPPKPKDTLMISRTPEVTCVVVDVSHEDPEVK |
| | | FNWYVDGVEVHNAKTKPREEQYNSTYRVVSVLTVLHQDWL |
| | | NGKEYKCKVSNKGLPSSIEKTISKAKGQPREPQVYTLPPS |
| | | REEMTKNQVSLTCLVKGFYPSDIAVEWESNGQPENNYKTT |
| | | PPVLDSDGSFFLYSKLTVDKSRWQQGNVFSCSVLHEALHA |
| | | HYTRKELSLSP* |
| 2 | Crovalimab | DIQMTQSPSSLSASVGDRVTITCRASQGISSSLAWYQQKP |
| | light chain | GKAPKLLIYGASETESGVPSRFSGSGSGTDFTLTISSLQP |
| | | EDFATYYCQNTKVGSSYGNTFGGGTKVEIKRTVAAPSVFI |
| | | FPPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC* |
| 3 | heavy chain | QVQLVESGGGLVQPGRSLRLSCAASGFTVHSSYYMAWVRQ |
| | variable | APGKGLEWVGAIFTGSGAEYKAEWAKGRVTISKDTSKNQV |
| | domain (VH) | VLTMTNMDPVDTATYYCASDAGYDYPTHAMHYWGQGTLVT |
| | of 305LO15 | VSS* |
| 4 | modified | ASTKGPSVFPLAPSSKSTSGGTAALGCLVKDYFPEPVTVS |
| | human IgG1 | WNSGALTSGVHTFPAVLQSSGLYSLSSVVTVPSSSLGTQT |
| | heavy chain | YICNVNHKPSNTKVDKKVEPKSCDKTHTCPPCPAPELRRG |
| | constant | PKVFLFPPKPKDTLMISRTPEVTCVVVDVSHEDPEVKFNW |
| | domain (CH) | YVDGVEVHNAKTKPREEQYNSTYRVVSVLTVLHQDWLNGK |
| | variant | EYKCKVSNKGLPSSIEKTISKAKGQPREPQVYTLPPSREE |
| | SG115 | MTKNQVSLTCLVKGFYPSDIAVEWESNGQPENNYKTTPPV |
| | | LDSDGSFFLYSKLTVDKSRWQQGNVFSCSVLHEALHAHYT |
| | | RKELSLSP* |
| 5 | light chain | DIQMTQSPSSLSASVGDRVTITCRASQGISSSLAWYQQKP |
| | variable | GKAPKLLIYGASETESGVPSRFSGSGSGTDFTLTISSLQP |
| | domain (VL) | EDFATYYCQNTKVGSSYGNTFGGGTKVEIK* |
| | of 305LO15 | |
| 6 | a human light | RTVAAPSVFIFPPSDEQLKSGTASVVCLLNNFYPREAKVQ |
| | chain | WKVDNALQSGNSQESVTEQDSKDSTYSLSSTLTLSKADYE |
| | constant | KHKVYACEVTHQGLSSPVTKSFNRGEC* |
| | domain (CL) | |
| | (SK1) | |

1. A method of treating Guillain-Barre Syndrome (GBS),
comprising intravenously administering a loading dose (IVD) of 1500 mg of an anti-C5 antibody once,
followed by subcutaneously administering at least one dose (SCD) of 340 mg of the anti-C5 antibody to the subject.

2. The method according to claim 1, wherein the at least one SCD of 340 mg is administered at least once to the subject one day to three weeks after the start of the IVD.

3. The method according to claim 2, wherein intravenous immunoglobulin (IVIg) is administered to the subject in combination with the anti-C5 antibody.

4. The method according to claim 3, wherein a dose of 400 mg/kg of IVIg is intravenously administered to the subject at least once.

5. The method according to claim 4, wherein the dose of 400 mg/kg of IVIg is intravenously administered daily to the subject on the same day as the IVD, one day, two days, three days and four days after the IVD.

6. The method according to claim 2, wherein the at least one SCD of 340 mg of the anti-C5 antibody is administered once to the subject one day after the start of the IVD of the anti-C5 antibody.

7. The method according to claim 1, wherein at least one additional SCD of 340 mg of the anti-C5 antibody is administered once to the subject one week or two weeks after the start of the IVD of the anti-C5 antibody.

8. The method according to claim 1, wherein an additional SCD (SCD1) of 340 mg of the anti-C5 antibody is subcutaneously administered to the subject one week after the start of the IVD, and wherein an additional SCD (SCD2) of 340 mg of the anti-C5 antibody is subcutaneously administered to the subject two weeks after the start of the IVD.

9. The method according to claim 8, wherein the subject is subcutaneously administered an SCD of 340 mg of the anti-C5 antibody at least once weekly over one to three weeks after the start of the IVD.

10. A method of treating GBS in a subject, comprising:
(i) intravenously administering a loading dose of 1500 mg of an anti-C5 antibody (IVD) to the subject once;
(ii) subcutaneously administering a first dose of 340 mg of the anti-C5 antibody to the subject one day after the start of the IVD; and
(iii) subcutaneously administering a dose of 340 mg of the anti-C5 antibody to the subject once weekly over one to three weeks after the start of the IVD.

11. The method according to claim 10, wherein the anti-C5 antibody is used in combination with IVIg, wherein 400 mg/kg IVIg is intravenously administered:
(a) once on the same day as the IVD; and
(b) daily on one day, two days, three days and four days after the start of the IVD.

12. The method according to claim 1 or 11, wherein the subject received prior treatment with at least one pharmacological product useful for the treatment of GBS wherein the IVD is administered to the subject after the final dose of the pharmacological product.

13. The method according to claim 1 or 11, wherein the subject has a body weight above 100 kg.

14. The method according to claim 1 or 11, wherein a biological sample from the subject is determined to have an anti-C5 antibody concentration that is 100 μg/ml or more.

15. The method according to claim 1 or 11, wherein the anti-C5 antibody is crovalimab.

16. A method of treating GBS in a subject, preferably in a subject with a body weight over 100 kg, wherein the method comprises the consecutive steps of administering to the subject:
(i) an intravenously administered loading dose of 1500 mg of the anti-C5 antibody (IVD) to the subject once and an intravenously administered dose of 400 mg/kg of IVIg once on the same day;
(ii) a subcutaneously administered maintenance dose of 340 mg of the anti-C5 antibody to the subject one day after the start of the IVD and an intravenously administered dose of 400 mg/kg of IVIg on the same day; and
